SEQUENCE LISTING (20 sequences)

SEQ ID NO: 1 - protein, 456 aa, Homo sapiens
  MISC_FEATURE (1)..(456): golimumab heavy chain (HC)
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, Homo sapiens
  MISC_FEATURE (1)..(215): golimumab light chain (LC)
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 126 aa, Homo sapiens
  MISC_FEATURE (1)..(126): golimumab variable heavy chain region
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 4 - protein, 111 aa, Homo sapiens
  MISC_FEATURE (1)..(111): golimumab variable light chain region
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTV

SEQ ID NO: 5 - protein, 5 aa, Homo sapiens
  MISC_FEATURE (1)..(5): golimumab complementarity determining region heavy chain 1 (CDRH1)
SYAMH

SEQ ID NO: 6 - protein, 17 aa, Homo sapiens
  MISC_FEATURE: golimumab complementarity determining region heavy chain 2 (CDRH2)
FMSYDGSNKKYADSVKG

SEQ ID NO: 7 - protein, 17 aa, Homo sapiens
  MISC_FEATURE (1)..(17): golimumab complementarity determining region heavy chain 3 (CDRH3)
DRGIAAGGNYYYYGMDV

SEQ ID NO: 8 - protein, 11 aa, Homo sapiens
  MISC_FEATURE (1)..(11): golimumab complementarity determining region light chain 1 (CDRL1)
RASQSVYSYLA

SEQ ID NO: 9 - protein, 7 aa, Homo sapiens
  MISC_FEATURE (1)..(7): golimumab complementarity determining region light chain 2 (CDRL2)
DASNRAT

SEQ ID NO: 10 - protein, 10 aa, Homo sapiens
  MISC_FEATURE (1)..(10): golimumab complementarity determining region light chain 3 (CDRL3)
QQRSNWPPFT

SEQ ID NO: 11 - protein, 449 aa, Homo sapiens
  MISC_FEATURE: sirukumab heavy chain (HC)
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 213 aa, Homo sapiens
  MISC_FEATURE: sirukumab light chain (LC)
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, Homo sapiens
  MISC_FEATURE: sirukumab variable heavy chain region
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 14 - protein, 109 aa, Homo sapiens
  MISC_FEATURE: sirukumab variable light chain region
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIKRTV

SEQ ID NO: 15 - protein, 5 aa, Homo sapiens
  MISC_FEATURE: sirukumab complementarity determining region heavy chain 1 (CDRH1)
PFAMS

SEQ ID NO: 16 - protein, 17 aa, Homo sapiens
  MISC_FEATURE: sirukumab complementarity determining region heavy chain 2 (CDRH2)
KISPGGSWTYYSDTVTG

SEQ ID NO: 17 - protein, 10 aa, Homo sapiens
  MISC_FEATURE: sirukumab complementarity determining region heavy chain 3 (CDRH3)
QLWGYYALDI

SEQ ID NO: 18 - protein, 10 aa, Homo sapiens
  MISC_FEATURE: sirukumab complementarity determining region light chain 1 (CDRL1)
SASISVSYMY

SEQ ID NO: 19 - protein, 7 aa, Homo sapiens
  MISC_FEATURE: sirukumab complementarity determining region light chain 2 (CDRL2)
DMSNLAS

SEQ ID NO: 20 - protein, 9 aa, Homo sapiens
  MISC_FEATURE: sirukumab complementarity determining region light chain 3 (CDRL3)
MQWSGYPYT

(19) United States
(12) Patent Application Publication
(10) Pub. No.: US 2021/0318334 A1
(43) Pub. Date: Oct. 14, 2021

(54) Materials and Methods for Improved Prediction of IGG Therapeutic Protein Exposure

(71) Applicant: Janssen Biotech, Inc., Horsham, PA (US)

(72) Inventors: Yang Chen, Ambler, PA (US); Jocelyn Leu, Ambler, PA (US); Yan Xu, Lansdale, PA (US); Zhenhua Xu, Berwyn, PA (US)

(21) Appl. No.: 17/229,124

(22) Filed: Apr. 13, 2021

Related U.S. Application Data
(60) Provisional application No. 63/009,536, filed on Apr. 14, 2020.
(60) Provisional application No. 63/009,532, filed on Apr. 14, 2020.
(60) Provisional application No. 63/009,527, filed on Apr. 14, 2020.
(60) Provisional application No. 63/009,523, filed on Apr. 14, 2020.
(60) Provisional application No. 63/009,521, filed on Apr. 14, 2020.

(51) Int. Cl. G01N 33/68; C07K 16/28

(52) U.S. Cl. CPC G01N 33/6854; G01N 2333/5412; G01N 2333/525; C07K 16/2866

(57) ABSTRACT

Methods for predicting pharmacokinetics of an Immunoglobulin G (IgG) therapeutic protein based on a patient's level of rheumatoid factor (RF), anti-citrullinated protein antibodies (ACPA), or RF and ACPA. Methods disclosed herein also relate to methods for determining the therapeutically effective dose of an IgG therapeutic protein based on the levels of RF, ACPA, or RF and ACPA.

### CROSS-REFERENCE TO RELATED APPLICATIONS

This application claims priority to U.S. Provisional Patent Application Ser. No. 63/009,521, filed Apr. 14, 2020, U.S. Provisional Patent Application Ser. No. 63/009,523, filed Apr. 14, 2020, U.S. Provisional Patent Application Ser. No. 63/009,527, filed Apr. 14, 2020, U.S. Provisional Patent Application Ser. No. 63/009,532, filed Apr. 14, 2020, and U.S. Provisional Patent Application Ser. No. 63/009,536, filed Apr. 14, 2020, each of which is incorporated by reference herein in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “JBI6284USNP1 Sequences.txt”, creation date of Apr. 9, 2021 and having a size of 21 kb. The sequence listing submitted via EFS-Web is part of the specification and is herein incorporated by reference in its entirety.

### FIELD OF THE INVENTION

The present invention relates to methods for predicting pharmacokinetics of an Immunoglobulin G (IgG) therapeutic protein based on a patient's level of rheumatoid factor (RF), anti-citrullinated protein antibodies (ACPA), or RF and ACPA. The present invention also relates to methods for determining the therapeutically effective dose of an IgG therapeutic protein based on the level of RF, ACPA, or RF and ACPA.

### BACKGROUND OF THE INVENTION

Rheumatoid factor (RF) and Anti-Citrullinated Protein Antibodies (ACPA) are autoantibodies directed against the Fc portion of immunoglobulin G (IgG) and autoantibodies directed against peptides or proteins that are citrullinated, respectively. High levels of RF and ACPA in the blood are often associated with autoimmune diseases, e.g., rheumatoid arthritis (RA), lupus, and Sjögren's syndrome (Ingegnoli et al. Dis Markers. 2013; 35(6): 727-734). High levels of RF may also appear in other diseases including viral, bacterial, and parasitic infections (Posnett and Edinger, J Exp Med. 1997 May 19; 185(10): 1721-1723). RF and ACPA have been used as diagnostic markers for RA and several studies have attempted to correlate the presence or level of RF and/or ACPA to a clinical response in RA patients treated with anti-TNF agents, e.g., Potter et al., Ann Rheum Dis, 2009, 68:69-74; Bobbio-Pallavicini et al., Ann Rheum Dis, 2007, 66:302-307; Hyrich et al., Rheumatology, 2006, 45, 1558-1565; Klaasen et al., Rheumatology, 2011, 50:1487-1493). The studies, however, generated conflicting results and failed to establish a clear association between the level of RF and/or ACPA and a response in patients with RA. There is also a report related to discriminating or categorizing RA patients as likely to respond to anti-tumor necrosis factor (anti-TNF) antibodies or anti-TNF antibody fragments based on a patient's level of RF and/or ACPA (U.S. Pat. App. No.: 2017/0328897).

Thus, there is a need for improved materials and methods for predicting pharmacokinetics of an Immunoglobulin G (IgG) therapeutic protein.

### SUMMARY OF THE INVENTION

The general and preferred embodiments are defined, respectively, by the independent and dependent claims appended hereto, which for the sake of brevity are incorporated by reference herein. Other embodiments, features, and advantages of the various aspects of the invention will become apparent from the detailed description below taken in conjunction with the appended drawing figures. Studies disclosed herein show that RF and ACPA levels have an impact on the pharmacokinetics (PK) of IgG therapeutic proteins. The findings of these studies may be used to improve treatments for patients with immune disorders, autoimmune diseases, non-immune-mediated diseases, and/or infections, and the like.

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA.

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA, wherein the reference value is 15 international units per milliliter (IU/mL) for RF and the reference value is 20 IU/ml for ACPA.

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA, wherein the reference value is 15 international units per milliliter (IU/mL) for RF and the reference value is 20 IU/ml for ACPA, and wherein the predicted exposure of the IgG therapeutic protein is a predicted steady state serum trough concentration, a predicted steady state serum peak concentration, or a predicted area under the curve concentration-time profile of the IgG therapeutic protein.

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA, wherein the reference value is 15 international units per milliliter (IU/mL) for RF and the reference value is 20 IU/ml for ACPA, and wherein the predicted steady state serum trough concentration, the predicted steady state serum peak concentration, or the predicted area under the curve concentration-time profile of the IgG therapeutic protein is about 20-22% lower in a patient positive for RF compared to a patient negative for RF.

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA, wherein the reference value is 15 international units per milliliter (IU/mL) for RF and the reference value is 20 IU/ml for ACPA, and wherein the predicted steady state serum trough concentration, the predicted steady state serum peak concentration, or the predicted area under the curve concentration-time profile of the IgG therapeutic protein is about 2-20% lower in a patient positive for ACPA compared to a patient negative for ACPA.

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA, wherein the reference value is 15 international units per milliliter (IU/mL) for RF and the reference value is 20 IU/ml for ACPA, and wherein the IgG therapeutic protein is selected from the group consisting of: an anti-tumor necrosis factor (anti-TNF) antibody, and anti-TNF Fc-fusion protein, an anti-interleukin-6 (anti-IL-6) antibody, and an anti-IL-6 receptor (IL-6R) antibody.

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA, wherein the reference value is 15 international units per milliliter (IU/mL) for RF and the reference value is 20 IU/ml for ACPA, and wherein the anti-TNF antibody is selected from the group consisting of SIMPONI® (golimumab), REMICADE® (infliximab), and HUMIRA® (adalimumab).

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA, wherein the reference value is 15 international units per milliliter (IU/mL) for RF and the reference value is 20 IU/ml for ACPA, and wherein the anti-TNF antibody is SIMPONI® (golimumab).

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA.

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA, wherein the reference value is 15 international units per milliliter (IU/mL) for RF and the reference value is 20 IU/ml for ACPA.

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA, wherein the predicted exposure of the IgG therapeutic protein is a predicted steady state serum trough concentration, a predicted steady state serum trough concentration, or a predicted area under the curve concentration-time profile of the IgG therapeutic protein.

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA, wherein the predicted exposure of the IgG therapeutic protein is a predicted steady state serum trough concentration, a predicted steady state serum trough concentration, or a predicted area under the curve concentration-time profile of the IgG therapeutic protein, and wherein the predicted steady state serum trough concentration, the predicted steady state serum trough concentration, or the predicted area under the curve concentration-time profile of the IgG therapeutic protein is about 17-29% lower in a patient positive for RF and ACPA compared to a patient negative for RF and ACPA.

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA, wherein the IgG therapeutic protein is selected from the group consisting of: an anti-tumor necrosis factor (anti-TNF) antibody, and anti-TNF Fc-fusion protein, an anti-interleukin-6 (anti-IL-6) antibody, and an anti-IL-6 receptor (IL-6R) antibody.

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA, wherein the IgG therapeutic protein is an anti-TNF antibody selected from the group consisting of SIMPONI® (golimumab), REMICADE® (infliximab), and HUMIRA® (adalimumab).

In certain embodiments, the present invention provides a method for predicting exposure of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA, wherein the IgG therapeutic protein is the anti-TNF antibody is SIMPONI® (golimumab).

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF compared to a patient negative for RF or predicting the serum trough concentration of the IgG therapeutic protein will be lower in a patient positive for ACPA compared to a patient negative for ACPA. In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF compared to a patient negative for RF or predicting the serum trough concentration of the IgG therapeutic protein will be lower in a patient positive for ACPA compared to a patient negative for ACPA, wherein the reference value is 15 international units per milliliter (IU/mL) for RF and the reference value is 20 IU/ml for ACPA.

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF compared to a patient negative for RF or predicting the serum trough concentration of the IgG therapeutic protein will be lower in a patient positive for ACPA compared to a patient negative for ACPA, wherein the predicted serum trough concentration of the IgG therapeutic protein is a predicted steady state serum trough concentration of the IgG therapeutic protein.

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF compared to a patient negative for RF or predicting the serum trough concentration of the IgG therapeutic protein will be lower in a patient positive for ACPA compared to a patient negative for ACPA, wherein the predicted serum trough concentration of the IgG therapeutic protein is a predicted steady state serum trough concentration of the IgG therapeutic protein, and wherein the predicted steady state serum trough concentration is about 20-22% lower in a patient positive for RF compared to a patient negative for RF.

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.)

comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF compared to a patient negative for RF or predicting the serum trough concentration of the IgG therapeutic protein will be lower in a patient positive for ACPA compared to a patient negative for ACPA, wherein the predicted serum trough concentration of the IgG therapeutic protein is a predicted steady state serum trough concentration of the IgG therapeutic protein, and wherein the predicted steady state serum trough concentration is about 2-20% lower in a patient positive for ACPA compared to a patient negative for ACPA.

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF compared to a patient negative for RF or predicting the serum trough concentration of the IgG therapeutic protein will be lower in a patient positive for ACPA compared to a patient negative for ACPA, wherein the IgG therapeutic protein is selected from the group consisting of: an anti-tumor necrosis factor (anti-TNF) antibody, and anti-TNF Fc-fusion protein, an anti-interleukin-6 (anti-IL-6) antibody, and an anti-IL-6 receptor (IL-6R) antibody.

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF compared to a patient negative for RF or predicting the serum trough concentration of the IgG therapeutic protein will be lower in a patient positive for ACPA compared to a patient negative for ACPA, wherein the IgG therapeutic protein is an anti-TNF antibody selected from the group consisting of SIMPONI® (golimumab), REMICADE® (infliximab), and HUMIRA® (adalimumab).

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF compared to a patient negative for RF or predicting the serum trough concentration of the IgG therapeutic protein will be lower in a patient positive for ACPA compared to a patient negative for ACPA, wherein the IgG therapeutic protein is the anti-TNF antibody is SIMPONI® (golimumab).

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF and positive for ACPA compared to a patient negative for RF and negative for ACPA.

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF and positive for ACPA compared to a patient negative for RF and negative for ACPA, wherein the reference value is 15 international units per milliliter (IU/mL) for RF and the reference value is 20 IU/ml for ACPA.

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF and positive for ACPA compared to a patient negative for RF and negative for ACPA, wherein the predicted serum trough concentration of the IgG therapeutic protein is a predicted steady state serum trough concentration of the IgG therapeutic protein.

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF and positive for ACPA compared to a patient negative for RF and negative for ACPA, wherein the predicted serum trough concentration of the IgG therapeutic protein is a predicted steady state serum trough concentration of the IgG therapeutic protein, and wherein the predicted steady state serum trough concentration is about 17-29% lower in a patient positive for RF and ACPA compared to a patient negative for RF and ACPA.

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF and positive for ACPA compared to a patient negative for RF and negative for ACPA, wherein the predicted serum trough concentration of the IgG therapeutic protein is a predicted steady state serum trough concentration of the IgG therapeutic protein, and wherein the IgG therapeutic protein is selected from the group consisting of: an anti-tumor necrosis factor (anti-TNF) antibody, and anti-TNF Fc-fusion protein, an anti-interleukin-6 (anti-IL-6) antibody, and an anti-IL-6 receptor (IL-6R) antibody.

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF and positive for ACPA compared to a patient negative for RF and negative for ACPA, wherein the predicted serum trough concentration of the IgG therapeutic protein is a predicted steady state serum trough concentration of the IgG therapeutic protein, and wherein the IgG therapeutic protein is an anti-TNF antibody selected from the group consisting of SIMPONI® (golimumab), REMICADE® (infliximab), and HUMIRA® (adalimumab).

In certain embodiments, the present invention provides a method for predicting serum trough concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum trough concentration of the IgG therapeutic protein will be lower in a patient positive for RF and positive for ACPA compared to a patient negative for RF and negative for ACPA, wherein the predicted serum trough concentration of the IgG therapeutic protein is a predicted steady state serum trough concentration of the IgG therapeutic protein, and wherein the IgG therapeutic protein is the anti-TNF antibody is SIMPONI® (golimumab).

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum peak concentration of the IgG therapeutic protein will be lower in a patient positive for RF compared to a patient negative for RF or predicting the serum peak concentration of the IgG therapeutic protein will be lower in a patient positive for ACPA compared to a patient negative for ACPA.

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.)

comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum peak concentration of the IgG therapeutic protein will be lower in a patient positive for RF compared to a patient negative for RF or predicting the serum peak concentration of the IgG therapeutic protein will be lower in a patient positive for ACPA compared to a patient negative for ACPA, wherein the reference value is 15 international units per milliliter (IU/mL) for RF and the reference value is 20 IU/ml for ACPA.

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum peak concentration of the IgG therapeutic protein will be lower in a patient positive for RF compared to a patient negative for RF or predicting the serum peak concentration of the IgG therapeutic protein will be lower in a patient positive for ACPA compared to a patient negative for ACPA, wherein the predicted serum peak concentration of the IgG therapeutic protein is a predicted steady state serum peak concentration of the IgG therapeutic protein.

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.)

comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum peak concentration of the IgG therapeutic protein will be lower in a patient positive for RF compared to a patient negative for RF or predicting the serum peak concentration of the IgG therapeutic protein will be lower in a patient positive for ACPA compared to a patient negative for ACPA, wherein the predicted serum peak concentration of the IgG therapeutic protein is a predicted steady state serum peak concentration of the IgG therapeutic protein, and wherein the predicted steady state serum peak concentration is about 20-22% lower in a patient positive for RF compared to a patient negative for RF.

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA;) predicting the serum peak concentration of the IgG therapeutic protein will be lower in a patient positive for RF compared to a patient negative for RF or predicting the serum peak concentration of the IgG therapeutic protein will be lower in a patient positive for ACPA compared to a patient negative for ACPA, wherein the predicted serum peak concentration of the IgG therapeutic protein is a predicted steady state serum peak concentration of the IgG therapeutic protein, and wherein the predicted steady state serum peak concentration of the IgG therapeutic protein is about 2-20% lower in a patient positive for ACPA compared to a patient negative for ACPA.

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the serum peak concentration of the IgG therapeutic protein will be lower in a patient positive for RF compared to a patient negative for RF or predicting the serum peak concentration of the IgG therapeutic protein will be lower in a patient positive for ACPA compared to a patient negative for ACPA, wherein the predicted serum peak concentration of the IgG therapeutic protein is a predicted steady state serum peak concentration of the IgG therapeutic protein, wherein the IgG therapeutic protein is selected from the group consisting of: an anti-tumor necrosis factor (anti-TNF) antibody, and anti-TNF Fc-fusion protein, an anti-interleukin-6 (anti-IL-6) antibody, and an anti-IL-6 receptor (IL-6R) antibody.

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA;) predicting the serum peak concentration of the IgG therapeutic protein will be lower in a patient positive for RF compared to a patient negative for RF or predicting the serum peak concentration of the IgG therapeutic protein will be lower in a patient positive for ACPA compared to a patient negative for ACPA, wherein the predicted serum peak concentration of the IgG therapeutic protein is a predicted steady state serum peak concentration of the IgG therapeutic protein, wherein the IgG therapeutic protein is and anti-TNF antibody selected from the group consisting of SIMPONI® (golimumab), REMICADE® (infliximab), and HUMIRA® (adalimumab).

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA;) predicting the serum peak concentration of the IgG therapeutic protein will be lower in a patient positive for RF compared to a patient negative for RF or predicting the serum peak concentration of the IgG therapeutic protein will be lower in a patient positive for ACPA compared to a patient negative for ACPA, wherein the predicted serum peak concentration of the IgG therapeutic protein is a predicted steady state serum peak concentration of the IgG therapeutic protein, wherein the IgG therapeutic protein is the anti-TNF antibody SIMPONI® (golimumab).

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum peak concentration of the IgG therapeutic protein will be lower in a patient positive for RF and positive for ACPA compared to a patient negative for RF and negative for ACPA.

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum peak concentration of the IgG therapeutic protein will be lower in a patient positive for RF and positive for ACPA compared to a patient negative for RF and negative for ACPA, wherein the reference value is 15 international units per milliliter (IU/mL) for RF and the reference value is 20 IU/ml for ACPA.

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum peak concentration of the IgG therapeutic protein will be lower in a patient positive for RF and positive for ACPA compared to a patient negative for RF and negative for ACPA, wherein the predicted serum peak concentration of the IgG therapeutic protein is a predicted steady state serum peak concentration of the IgG therapeutic protein.

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum peak concentration of the IgG therapeutic protein will be lower in a patient positive for RF and positive for ACPA compared to a patient negative for RF and negative for ACPA, wherein the predicted serum peak concentration of the IgG therapeutic protein is a predicted steady state serum peak concentration of the IgG therapeutic protein, and wherein the predicted steady state serum peak concentration of the IgG therapeutic protein is about 17-29% lower in a patient positive for RF and ACPA compared to a patient negative for RF and ACPA.

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum peak concentration of the IgG therapeutic protein will be lower in a patient positive for RF and positive for ACPA compared to a patient negative for RF and negative for ACPA, wherein the predicted serum peak concentration of the IgG therapeutic protein is a predicted steady state serum peak concentration of the IgG therapeutic protein, and wherein the IgG therapeutic protein is selected from the group consisting of: an anti-tumor necrosis factor (anti-TNF) antibody, and anti-TNF Fc-fusion protein, an anti-interleukin-6 (anti-IL-6) antibody, and an anti-IL-6 receptor (IL-6R) antibody.

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum peak concentration of the IgG therapeutic protein will be lower in a patient positive for RF and positive for ACPA compared to a patient negative for RF and negative for ACPA, wherein the predicted serum peak concentration of the IgG therapeutic protein is a predicted steady state serum peak concentration of the IgG therapeutic protein, and wherein the IgG therapeutic protein is an anti-TNF antibody selected from the group consisting of SIMPONI® (golimumab), REMICADE® (infliximab), and HUMIRA® (adalimumab).

In certain embodiments, the present invention provides a method for predicting serum peak concentration of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the serum peak concentration of the IgG therapeutic protein will be lower in a patient positive for RF and positive for ACPA compared to a patient negative for RF and negative for ACPA, wherein the predicted serum peak concentration of the IgG therapeutic protein is a predicted steady state serum peak concentration of the IgG therapeutic protein, and wherein the IgG therapeutic protein is anti-TNF antibody SIMPONI® (golimumab).

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the AUC of the IgG therapeutic protein will be lower in a patient positive for RF compared to a patient negative for RF or predicting the AUC of the IgG therapeutic protein will be lower in a patient positive for ACPA compared to a patient negative for ACPA.

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the AUC of the IgG therapeutic protein will be lower in a patient positive for RF compared to a patient negative for RF or predicting the AUC of the IgG therapeutic protein will be lower in a patient positive for ACPA compared to a patient negative for ACPA, wherein the reference value is 15 international units per milliliter (IU/mL) for RF and the reference value is 20 IU/ml for ACPA.

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the AUC of the IgG therapeutic protein will be lower in a patient positive for RF compared to a patient negative for RF or predicting the AUC of the IgG therapeutic protein will be lower in a patient positive for ACPA compared to a patient negative for ACPA, wherein the predicted AUC of the IgG therapeutic protein is about 20-22% lower in a patient positive for RF compared to a patient negative for RF.

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the AUC of the IgG therapeutic protein will be lower in a patient positive for RF compared to a patient negative for RF or predicting the AUC of the IgG therapeutic protein will be lower in a patient positive for ACPA compared to a patient negative for ACPA, wherein the predicted AUC of the IgG therapeutic protein is about 2-20% lower in a patient positive for ACPA compared to a patient negative for ACPA.

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the AUC of the IgG therapeutic protein will be lower in a patient positive for RF compared to a patient negative for RF or predicting the AUC of the IgG therapeutic protein will be lower in a patient positive for ACPA compared to a patient negative for ACPA, wherein the IgG therapeutic protein is selected from the group consisting of: an anti-tumor necrosis factor (anti-TNF) antibody, and anti-TNF Fc-fusion protein, an anti-interleukin-6 (anti-IL-6) antibody, and an anti-IL-6 receptor (IL-6R) antibody.

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the AUC of the IgG therapeutic protein will be lower in a patient positive for RF compared to a patient negative for RF or predicting the AUC of the IgG therapeutic protein will be lower in a patient positive for ACPA compared to a patient negative for ACPA, wherein the IgG therapeutic protein is an anti-TNF antibody selected from the group consisting of SIMPONI® (golimumab), REMICADE® (infliximab), and HUMIRA® (adalimumab).

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the AUC of the IgG therapeutic protein will be lower in a patient positive for RF compared to a patient negative for RF or predicting the AUC of the IgG therapeutic protein will be lower in a patient positive for ACPA compared to a patient negative for ACPA, wherein the IgG therapeutic protein is the anti-TNF antibody SIMPONI® (golimumab).

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the AUC of the IgG therapeutic protein will be lower in a patient positive for RF and positive for ACPA compared to a patient negative for RF and negative for ACPA.

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the AUC of the IgG therapeutic protein will be lower in a patient positive for RF and positive for ACPA compared to a patient negative for RF and negative for ACPA, wherein the reference value is 15 international units per milliliter (IU/mL) for RF and the reference value is 20 IU/ml for ACPA.

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the AUC of the IgG therapeutic protein will be lower in a patient positive for RF and positive for ACPA compared to a patient negative for RF and negative for ACPA, wherein the AUC of the IgG therapeutic protein is about 17-29% lower in a patient positive for RF and ACPA compared to a patient negative for RF and ACPA.

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the AUC of the IgG therapeutic protein will be lower in a patient positive for RF and positive for ACPA compared to a patient negative for RF and negative for ACPA, wherein the IgG therapeutic protein is selected from the group consisting of: an anti-tumor necrosis factor (anti-TNF) antibody, and anti-TNF Fc-fusion protein, an anti-interleukin-6 (anti-IL-6) antibody, and an anti-IL-6 receptor (IL-6R) antibody.

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the AUC of the IgG therapeutic protein will be lower in a patient positive for RF and positive for ACPA compared to a patient negative for RF and negative for ACPA, wherein the IgG therapeutic protein is anti-TNF antibody selected from the group consisting of SIMPONI® (golimumab), REMICADE® (infliximab), and HUMIRA® (adalimumab).

In certain embodiments, the present invention provides a method for predicting area under the curve concentration-time profile (AUC) of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA;) predicting the AUC of the IgG therapeutic protein will be lower in a patient positive for RF and positive for ACPA compared to a patient negative for RF and negative for ACPA, wherein the IgG therapeutic protein is the anti-TNF antibody SIMPONI® (golimumab).

In certain embodiments, the present invention provides a method for determining a therapeutically effective dose of an IgG therapeutic protein by predicting exposure of the IgG therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA; g.) determining the therapeutically effective dose of the IgG therapeutic protein should be administered at a higher dose in a patient positive for RF compared to a patient negative for RF or determining the therapeutically effective dose of the IgG therapeutic protein should be administered at a higher dose in a patient positive for ACPA compared to a patient negative for ACPA.

In certain embodiments, the present invention provides a method for determining a therapeutically effective dose of an IgG therapeutic protein by predicting exposure of the IgG therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA; g.) determining the therapeutically effective dose of the IgG therapeutic protein should be administered at a higher dose in a patient positive for RF compared to a patient negative for RF or determining the therapeutically effective dose of the IgG therapeutic protein should be administered at a higher dose in a patient positive for ACPA compared to a patient negative for ACPA, wherein the reference value is 15 international units per milliliter (IU/mL) for RF and the reference value is 20 IU/ml for ACPA.

In certain embodiments, the present invention provides a method for determining a therapeutically effective dose of an IgG therapeutic protein by predicting exposure of the IgG therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA; g.) determining the therapeutically effective dose of the IgG therapeutic protein should be administered at a higher dose in a patient positive for RF compared to a patient negative for RF or determining the therapeutically effective dose of the IgG therapeutic protein should be administered at a higher dose in a patient positive for ACPA compared to a patient negative for ACPA, wherein the predicted exposure of the IgG therapeutic protein is a predicted steady state serum trough concentration, a predicted steady state serum trough concentration, or a predicted area under the curve concentration-time profile of the IgG therapeutic protein.

In certain embodiments, the present invention provides a method for determining a therapeutically effective dose of an IgG therapeutic protein by predicting exposure of the IgG therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA; g.) determining the therapeutically effective dose of the IgG therapeutic protein should be administered at a higher dose in a patient positive for RF compared to a patient negative for RF or determining the therapeutically effective dose of the IgG therapeutic protein should be administered at a higher dose in a patient positive for ACPA compared to a patient negative for ACPA, wherein it is determined that the therapeutically effective dose of the IgG therapeutic protein should be administered at a dose about 20-22% higher in a patient positive for RF compared to a patient negative for RF, and wherein the patient negative for RF receives the recommended therapeutic dose for the IgG therapeutic protein.

In certain embodiments, the present invention provides a method for determining a therapeutically effective dose of an IgG therapeutic protein by predicting exposure of the IgG therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA; g.) determining the therapeutically effective dose of the IgG therapeutic protein should be administered at a higher dose in a patient positive for RF compared to a patient negative for RF or determining the therapeutically effective dose of the IgG therapeutic protein should be administered at a higher dose in a patient positive for ACPA compared to a patient negative for ACPA, wherein it is determined that the therapeutically effective dose of the IgG therapeutic protein should be administered at a dose about 2-20% higher in a patient positive for ACPA compared to a patient negative for ACPA, and wherein the patient negative for ACPA receives the recommended therapeutic dose for the IgG therapeutic protein.

In certain embodiments, the present invention provides a method for determining a therapeutically effective dose of an IgG therapeutic protein by predicting exposure of the IgG therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA; g.) determining the therapeutically effective dose of the IgG therapeutic protein should be administered at a higher dose in a patient positive for RF compared to a patient negative for RF or determining the therapeutically effective dose of the IgG therapeutic protein should be administered at a higher dose in a patient positive for ACPA compared to a patient negative for ACPA, wherein the IgG therapeutic protein is selected from the group consisting of: an anti-tumor necrosis factor (anti-TNF) antibody, and anti-TNF Fc-fusion protein, an anti-interleukin-6 (anti-IL-6) antibody, and an anti-IL-6 receptor (IL-6R) antibody.

In certain embodiments, the present invention provides a method for determining a therapeutically effective dose of an IgG therapeutic protein by predicting exposure of the IgG therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA;) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA; g.) determining the therapeutically effective dose of the IgG therapeutic protein should be administered at a higher dose in a patient positive for RF compared to a patient negative for RF or determining the therapeutically effective dose of the IgG therapeutic protein should be administered at a higher dose in a patient positive for ACPA compared to a patient negative for ACPA, wherein the IgG therapeutic protein is an anti-TNF antibody selected from the group consisting of SIMPONI® (golimumab), REMICADE® (infliximab), and HUMIRA® (adalimumab).

In certain embodiments, the present invention provides a method for determining a therapeutically effective dose of an IgG therapeutic protein by predicting exposure of the IgG therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA;) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA; g.) determining the therapeutically effective dose of the IgG therapeutic protein should be administered at a higher dose in a patient positive for RF compared to a patient negative for RF or determining the therapeutically effective dose of the IgG therapeutic protein should be administered at a higher dose in a patient positive for ACPA compared to a patient negative for ACPA, wherein the IgG therapeutic protein is the anti-TNF antibody SIMPONI® (golimumab).

In certain embodiments, the present invention provides a method for predicting a therapeutically effective dose of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA. g.) determining the therapeutically effective dose of the IgG therapeutic protein should be administered at a higher dose in a patient positive for RF and positive for ACPA compared to a patient negative for RF and negative for ACPA.

In certain embodiments, the present invention provides a method for predicting a therapeutically effective dose of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA. g.) determining the therapeutically effective dose of the IgG therapeutic protein should be administered at a higher dose in a patient positive for RF and positive for ACPA compared to a patient negative for RF and negative for ACPA, wherein the reference value is 15 international units per milliliter (IU/mL) for RF and the reference value is 20 IU/ml for ACPA.

In certain embodiments, the present invention provides a method for predicting a therapeutically effective dose of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA. g.) determining the therapeutically effective dose of the IgG therapeutic protein should be administered at a higher dose in a patient positive for RF and positive for ACPA compared to a patient negative for RF and negative for ACPA, wherein the predicted exposure of the IgG therapeutic protein is a predicted steady state serum trough concentration, a predicted steady state serum trough concentration, or a predicted area under the curve concentration-time profile of the IgG therapeutic protein.

In certain embodiments, the present invention provides a method for predicting a therapeutically effective dose of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA. g.) determining the therapeutically effective dose of the IgG therapeutic protein should be administered at a higher dose in a patient positive for RF and positive for ACPA compared to a patient negative for RF and negative for ACPA, wherein it is determined that the therapeutically effective dose of the IgG therapeutic protein should be administered at a dose about 17-29% higher in a patient positive for RF and ACPA compared to a patient negative for RF and ACPA.

In certain embodiments, the present invention provides a method for predicting a therapeutically effective dose of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA. g.) determining the therapeutically effective dose of the IgG therapeutic protein should be administered at a higher dose in a patient positive for RF and positive for ACPA compared to a patient negative for RF and negative for ACPA, wherein the IgG therapeutic protein is selected from the group consisting of: an anti-tumor necrosis factor (anti-TNF) antibody, and anti-TNF Fc-fusion protein, an anti-interleukin-6 (anti-IL-6) antibody, and an anti-IL-6 receptor (IL-6R) antibody.

In certain embodiments, the present invention provides a method for predicting a therapeutically effective dose of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA. g.) determining the therapeutically effective dose of the IgG therapeutic protein should be administered at a higher dose in a patient positive for RF and positive for ACPA compared to a patient negative for RF and negative for ACPA, wherein the IgG therapeutic protein is an anti-TNF antibody selected from the group consisting of SIMPONI® (golimumab), REMICADE® (infliximab), and HUMIRA® (adalimumab).

In certain embodiments, the present invention provides a method for predicting a therapeutically effective dose of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA. g.) determining the therapeutically effective dose of the IgG therapeutic protein should be administered at a higher dose in a patient positive for RF and positive for ACPA compared to a patient negative for RF and negative for ACPA, wherein the IgG therapeutic protein is the anti-TNF antibody SIMPONI® (golimumab).

In certain embodiments, the present invention provides a method for determining a therapeutically effective dose of an IgG therapeutic protein by predicting exposure of the IgG therapeutic protein in a human patient in need of treatment with the IgG therapeutic protein, the method comprising: a.) selecting the human patient in need of treatment with the IgG therapeutic protein; b.) determining a level of rheumatoid factor (RF) or a level of anti-citrullinated protein antibodies (ACPA) in a biological sample from the patient; c.) comparing the level of the RF in the sample to a reference value for RF or comparing the level of the ACPA in the sample to a reference value for ACPA; d.) determining if the patient is positive or negative for said RF or said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF or said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF compared to a patient negative for RF or predicting the exposure of the IgG therapeutic protein will be lower in a patient positive for ACPA compared to a patient negative for ACPA; g.) determining the therapeutically effective dose of the IgG therapeutic protein should be administered at a higher dose in a patient positive for RF compared to a patient negative for RF or determining the therapeutically effective dose of the IgG therapeutic protein should be administered at a higher dose in a patient positive for ACPA compared to a patient negative for ACPA; and, h.) administering the higher dose of the IgG therapeutic protein to the human patient in need of treatment with the IgG therapeutic protein.

In certain embodiments, the present invention provides a method for predicting a therapeutically effective dose of an immunoglobulin G (IgG) therapeutic protein in a human patient in need of treatment with the IgG therapeutic protein, the method comprising: a.) selecting the human patient in need of treatment with the IgG therapeutic protein; b.) determining a level of rheumatoid factor (RF) and a level of anti-citrullinated protein antibodies (ACPA) in a biological sample from the patient; c.) comparing the level of the RF in the sample to a reference value for RF and comparing the level of the ACPA in the sample to a reference value for ACPA; d.) determining if the patient is positive for said RF and positive for said ACPA, wherein the patient is positive for RF if the level of the RF in the sample is greater than or equal to (≥) the reference value for RF and the patient is positive for ACPA if the level of the ACPA in the sample is ≥the reference value for ACPA; e.) determining if the patient is negative for said RF and negative for said ACPA, wherein the patient is negative for RF if the level of the RF in the sample is less than (<) the reference value for RF and the patient is negative for ACPA if the level of the ACPA in the sample is <the reference value for ACPA; f.) predicting the exposure of the IgG therapeutic protein will be lower in a patient positive for RF and positive for ACPA compared to a patient negative for RF and negative for ACPA. g.) determining the therapeutically effective dose of the IgG therapeutic protein should be administered at a higher dose in a patient positive for RF and positive for ACPA compared to a patient negative for RF and negative for ACPA; and, h.) administering the higher dose of the IgG therapeutic protein to the human patient in need of treatment with the IgG therapeutic protein.

### DESCRIPTION OF THE FIGURES

FIG. 1 shows sirukumab steady state serum trough concentrations (μg/ml) at week 16 grouped by baseline rheumatoid factor (RF) status (Positive: RF ≥15 IU/mL vs Negative: RF<15 IU/mL). Q2w=every 2 weeks; Q4w=every 4 weeks.

FIG. 2 shows sirukumab steady state serum trough concentrations (μg/ml) at week 16 grouped by baseline anti-citrullinated protein antibodies (ACPA) status (Positive: ACPA ≥20 IU/mL versus Negative: ACPA <20 IU/mL). Q2w=every 2 weeks; Q4w=every 4 weeks.

FIG. 3 shows sirukumab steady state serum trough concentrations (μg/ml) at week 16 grouped by baseline rheumatoid factor (RF) status (Positive: RF ≥15 IU/mL vs Negative: RF<15 IU/mL) and methotrexate use. MTX=Methotrexate; Q2w=every 2 weeks; Q4w=every 4 weeks.

FIG. 4 shows sirukumab steady state serum trough concentrations (μg/ml) at week 16 grouped by baseline anti-citrullinated protein antibodies (ACPA) status (Positive: ACPA ≥20 IU/mL versus Negative: ACPA <20 IU/mL) and methotrexate use. MTX=Methotrexate; Q2w=every 2 weeks; Q4w=every 4 weeks.

FIG. 5 shows sirukumab steady state serum trough concentrations (μg/ml) at week 16 grouped by baseline rheumatoid factor (RF) status (Positive: RF ≥15 IU/mL vs Negative: RF<15 IU/mL) and baseline anti-citrullinated protein antibodies (ACPA) status (Positive: ACPA ≥20 IU/mL versus Negative: ACPA <20 IU/mL). Q2w=every 2 weeks; Q4w=every 4 weeks

FIG. 6 shows baseline rheumatoid factor (RF) levels grouped by baseline anti-citrullinated protein antibodies (ACPA) status (Positive: ACPA ≥20 IU/mL versus Negative: ACPA <20 IU/mL) in sirukumab Phase 3 Trials. STDY3002=SURROUND-D Trial; STDY3003=SURROUND-T Trial

FIG. 7 shows golimumab steady state serum trough concentrations (μg/ml) at week 24 grouped by baseline rheumatoid factor (RF) status (Positive: RF ≥15 IU/mL vs Negative: RF<15 IU/mL). MTX=Methotrexate; Q4w=every 4 weeks.

FIG. 8 shows golimumab steady state serum trough concentrations (μg/ml) at week 24 grouped by baseline anti-citrullinated protein antibodies (ACPA) status (Positive: ACPA ≥20 IU/mL versus Negative: ACPA <20 IU/mL). MTX=Methotrexate; Q4w=every 4 weeks.

FIG. 9 shows golimumab steady state serum trough concentrations (μg/ml) at week 24 grouped by baseline rheumatoid factor (RF) status (Positive: RF ≥15 IU/mL vs Negative: RF<15 IU/mL) and baseline anti-citrullinated protein antibodies (ACPA) status (Positive: ACPA ≥20 IU/mL versus Negative: ACPA <20 IU/mL).

FIG. 10 shows baseline rheumatoid factor (RF) levels grouped by baseline anti-citrullinated protein antibodies (ACPA) status (Positive: ACPA ≥20 IU/mL versus Negative: ACPA <20 IU/mL) in golimumab Phase 3 Trials. STDY05=GO-BEFORE Trial; STDY06=GO-FORWARD Trial.

### DESCRIPTION OF THE INVENTION

The effects of the levels of rheumatoid factor (RF) and anti-citrullinated protein antibodies (ACPA) on the pharmacokinetics (PK) of immunoglobulin G (IgG) therapeutic proteins were determined from 4 Phase III trials in patients with an autoimmune disease, i.e., active rheumatoid arthritis (RA). The data were pooled from the SIRROUND-D and SIRROUND-T clinical trials with sirukumab (Aletaha et al., Lancet. 2017, 389:1206-1217; Takeuchi et al., Ann Rheum Dis. 2017, 76:2001-2008) and the GO-BEFORE and GO-FORWARD clinical trials with golimumab (Emery et al., Arthritis Care Res (Hoboken). 2013 November; 65(11):1732-42; Genovese et al., J Rheumatol. 2012 June; 39(6):1185-91). Sirukumab and golimumab are human IgG monoclonal antibodies. Sirukumab (also known as CNTO 136) is an anti-interleukin-6 (anti-IL-6) antibody (Bartoli et al., Expert Rev Clin Immunol. 2018 July; 14(7):539-547) and golimumab (also known as SIMPONI®) is an anti-tumor necrosis factor (anti-TNF) antibody.

As used herein, “IgG therapeutic protein” or “IgG therapeutic proteins” refer to therapeutic monoclonal antibodies or fusion proteins that include the Fc domain (fragment crystallizable domain) of an IgG. Non-limiting examples of IgG therapeutic proteins that inhibit the IL-6 pathway include, e.g., the anti-interleukin-6 (anti-IL-6) antibody sirukumab (also known as CNTO 136) and the anti-interleukin-6 receptor (anti-IL-6R) antibodies ACTEMRA® (tocilizumab) (Bartoli et al., Expert Rev Clin Immunol. 2018 July; 14(7):539-547) and KEVZARA® (sarilumab) (Lamb and Deeks, Drugs. 2018 June; 78(9):929-940). Non-limiting examples of anti-TNF IgG therapeutic proteins include, e.g., anti-TNF antibodies such as SIMPONI® (golimumab), REMICADE® (infliximab) and HUMIRA® (adalimumab), and the soluble TNF receptor Fc-fusion protein ENBREL® (etanercept). For a review of TNF inhibitors, see, e.g., Lis et al., Arch Med Sci. 2014 Dec. 22; 10(6): 1175-1185.

As used herein, the term “rheumatoid factor” or “RF” refers to an autoantibody (i.e., an antibody produced by an organism in response to a constituent of its own tissues) that binds the Fc domain of IgG. These autoantibodies are often elevated in rheumatoid arthritis patients but can also be abnormally high in other pathologies, e.g., lupus, and Sjögren's syndrome (Ingegnoli et al. Dis Markers. 2013; 35(6): 727-734) and in viral, bacterial, and parasitic infections (Posnett and Edinger, J Exp Med. 1997 May 19; 185(10): 1721-1723). Assays for detecting RF are commercially available from different vendors, e.g., QUANTA FLASH® (chemiluminescent assays) for RF IgM and RF IgA, Inova Diagnostics (San Diego, Calif., US). The reference value used for RF positive is RF ≥15 IU/mL and the reference value used for RF negative is RF<15 IU/ml (Santos-Moreno et al., Medicine (Baltimore). 2019 February; 98(5): e14181).

As used herein, the terms “anti-citrullinated protein antibody,” or “ACPA” include autoantibodies specifically targeting one or more epitopes in a peptide, polypeptide, or protein sequence wherein during a post-translational modification one or more arginine residues have been converted into a citrulline residue. The presence or level of ACPA can be measured using natural or synthetic citrullinated peptides that are immunoreactive with ACPA. Assays for detecting ACPA are commercially available from different vendors, e.g., QUANTA FLASH® CCP3 (ELISA assay), Inova Diagnostics (San Diego, Calif., US). The reference value used for ACPA positive is ACPA ≥20 IU/ml and the reference value used for ACPA negative is ACPA <20 IU/ml (Santos-Moreno et al., Medicine (Baltimore). 2019 February; 98(5): e14181).

By the phrase “determining the level of” is meant an assessment of the degree of expression of a marker in a sample at the nucleic acid or protein level, using technology available to the skilled artisan to detect a sufficient portion of any marker expression product.

The terms “underexpress,” “underexpression,” “underexpressed,” or “down-regulated” interchangeably refer to a protein or nucleic acid that is transcribed or translated at a detectably lower level in a biological sample from a woman with autoimmune disease, in comparison to a biological sample from a woman without autoimmune disease. The term includes underexpression due to transcription, post transcriptional processing, translation, post-translational processing, cellular localization (e.g., organelle, cytoplasm, nucleus, cell surface), and RNA and protein stability, as compared to a control. Underexpression can be detected using conventional techniques for detecting mRNA (i.e., Q-PCR, RT-PCR, PCR, hybridization) or proteins (i.e., ELISA, immunohistochemical techniques). Underexpression can be 10%, 20%, 30%, 40%, 50%, 60%, 70%, 80%, 90% or less in comparison to a control. In some instances, underexpression is 1-, 2-, 3-, 4-, 5-, 6-, 7-, 8-, 9-, 10-fold or more lower levels of transcription or translation in comparison to a control.

The terms “overexpress,” “overexpression,” “overexpressed,” or “up-regulated” interchangeably refer to a protein or nucleic acid (RNA) that is transcribed or translated at a detectably greater level, usually in a biological sample from a woman with autoimmune disease, in comparison to a biological sample from a woman without autoimmune disease. The term includes overexpression due to transcription, post transcriptional processing, translation, post-translational processing, cellular localization (e.g., organelle, cytoplasm, nucleus, cell surface), and RNA and protein stability, as compared to a cell from a woman without autoimmune disease. Overexpression can be detected using conventional techniques for detecting mRNA (i.e., Q-PCR, RT-PCR, PCR, hybridization) or proteins (i.e., ELISA, immunohistochemical techniques). Overexpression can be 10%, 20%, 30%, 40%, 50%, 60%, 70%, 80%, 90% or more in comparison to a cell from a woman without autoimmune disease. In some instances, overexpression is 1-, 2-, 3-, 4-, 5-, 6-, 7-, 8-, 9-, 10-fold, or more higher levels of transcription or translation in comparison to a cell from a woman without autoimmune disease.

As used herein, the phrase “difference of the level” refers to differences in the quantity of a particular marker, such as a nucleic acid (e.g., microRNA, etc.) or a protein, in a sample as compared to a control or reference level. For example, the quantity of a particular biomarker may be present at an elevated amount or at a decreased amount in samples of patients with a disease compared to a reference level. In one embodiment, a “difference of a level” may be a difference between the quantity of a particular biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, a “difference of a level” may be a statistically significant difference between the quantity of a biomarker present in a sample as compared to a control. For example, a difference may be statistically significant if the measured level of the biomarker falls outside of about 1.0 standard deviations, about 1.5 standard deviations, about 2.0 standard deviations, or about 2.5 stand deviations of the mean of any control or reference group.

“Differentially increased expression” or “up regulation” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments there between compared to a comparator, or reference value.

“Differentially decreased expression” or “down regulation” refers to expression levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments there between compared to a comparator, or reference value.

As used herein, the terms “reference level,” “cut-off level,” or “threshold level” refer to predefined values for a given marker. A reference level is typically based on statistical analysis of a larger population or data set and is used for comparison to an individual subject or group of subjects. A person having ordinary skill in the art would understand how to determine a “reference level,” “cut-off level,” or “threshold level” for a given marker.

A “reference value” or “control value” as used herein may refer to a predetermined amount of a particular protein or nucleic acid that is detectable in a biological sample. In some embodiments, a reference value is suitable for the use of a method of the present invention, for comparing the amount of a protein or nucleic acid of interest that is present in a biological sample. An established sample serving as a reference control may provide an amount of the protein or nucleic acid of interest in the biological sample that is typical for an average, healthy person of reasonably matched background, e.g., gender, age, ethnicity, and medical history. A standard control value may vary depending on the protein or nucleic acid of interest and the nature of the sample (e.g., serum).

In certain embodiments, the reference value for RF level is 15 IU/mL, wherein RF positive is RF ≥15 IU/mL and RF negative is RF<15 IU/mL. In certain embodiments, the reference value for ACPA level is 20 IU/mL, wherein ACPA positive is ACPA ≥20 IU/mL and ACPA negative is ACPA <20 IU/mL.

In certain embodiments, an RF level corresponding to being positive for RF (e.g., RF ≥15 IU/mL) indicates a patient is predicted to have lower exposure to an IgG therapeutic protein than a patient that is negative for RF (e.g., RF<15 IU/mL). In certain embodiments, an ACPA level corresponding to being positive for ACPA (e.g., ACPA ≥20 IU/mL) indicates a patient with is predicted to have lower exposure to an IgG therapeutic protein than a patient that is negative for ACPA (e.g., ACPA <20 IU/mL). In certain embodiments, RF and ACPA levels corresponding to being positive for both RF (ie.g., RF ≥15 IU/mL) and ACPA (e.g., ACPA ≥20 IU/mL) indicates a patient is predicted to have lower exposure to an IgG therapeutic protein than a patient that is negative for RF (e.g., RF<15 IU/mL) and ACPA (e.g., ACPA <20 IU/mL).

In certain embodiments, the present invention advantageously enables a clinician to practice “personalized medicine” by guiding treatment decisions such that a specific therapeutically effective dose of an IgG therapeutic protein can be determined by predicting the pharmacokinetics of IgG therapeutic protein based on a patient's level of RF, ACPA, or RF and ACPA. The present invention also relates to methods for administering a therapeutically effective dose of IgG therapeutic proteins based on a patient's level of RF, ACPA, or RF and ACPA.

As used herein, the terms “therapeutically effective amount” or “therapeutically effective dose” refer to an amount capable of achieving a therapeutic effect in a subject in need thereof. For example, a therapeutically effective amount of an IgG therapeutic protein useful for treating an autoimmune disease or infection in a patient is an amount capable of preventing or relieving one or more symptoms associated with the autoimmune disease or infection. A therapeutically effective amount of IgG therapeutic protein may also be administered prophylactically in order to reduce the risk of developing the disease or infection or to delay the onset or recurrence of an event in progression of the disease or infection.

In certain embodiments, a therapeutically effective amount of IgG therapeutic protein may be administered to a patient with RA in a therapeutically effective amount for treating one or more symptoms associated with RA. In certain embodiments, a therapeutically effective amount of IgG therapeutic protein may be administered to a patient with a viral infection in a therapeutically effective amount for treating one or more symptoms associated with the viral infection.

As used herein, “recommended therapeutic dose” or “recommended therapeutic amount”, is the dose for the indication and/or route of administration on the label of the IgG therapeutic protein. For example, the dose on the label for SIMPONI ARIA® (golimumab) for intravenous use is “2 mg/kg intravenous infusion over 30 minutes at weeks 0 and 4, then every 8 weeks”. The dose on the label for SIMPONI® (golimumab) for subcutaneous use is “50 mg administered by subcutaneous injection once a month”. Intravenous SIMPONI ARIA® (golimumab) and subcutaneous SIMPONI® (golimumab) are currently approved for use in rheumatoid arthritis (RA), active psoriatic arthritis (PsA), and active ankylosing spondylitis (AS).

As used herein, “pharmacokinetics” of an IgG therapeutic protein refers to the exposure of the IgG therapeutic protein in a patient in need of treatment with the IgG therapeutic protein. As used herein, “exposure” refers to the level (concentration) achieved in the body of the patient. For example, exposure can be determined by different pharmacokinetic parameters, including, e.g., area under the curve concentration-time profiles (AUC), peak serum concentrations (Cₘₐₓ), or trough serum concentrations (Cₘᵢₙ). Peak and trough concentrations can be determined at single time points or can be determined at steady state. Steady state is when equilibrium occurs, and the peak and trough concentrations are the same with two or more successive doses. As used herein, exposure can be determined with serum or plasma concentrations. See, e.g., Ovacik and Kedan, Clin Transl Sci. 2018 November; 11(6): 540-552; Scheff et al., Pharm Res. 2011 May; 28(5): 1081-1089; Srinivas and Syed, Drugs R D. 2016 March; 16(1): 69-79; and the U.S. FDA Guidance Document for Content and Format of the Dosage and Administration Section of Labeling for Human Prescription Drug and Biological Products (March 2010). In certain embodiments, the exposure is determined by serum trough Cₘᵢₙ concentration of the IgG therapeutic protein. In certain embodiments, the exposure is determined by steady state serum trough concentration of the IgG therapeutic protein. In certain embodiments, the exposure is determined by peak serum concentrations (Cₘₐₓ) of the IgG therapeutic protein. In certain embodiments, the exposure is determined by steady state peak serum concentrations of the IgG therapeutic protein. In certain embodiments, the exposure is determined by area under the curve concentration-time profiles (AUC) of the IgG therapeutic protein.

As used herein, “monoclonal antibody” or “monoclonal antibodies” refer to whole monoclonal antibodies having two heavy chains and two light chains. The two heavy chains are linked together by disulfide bonds and each heavy chain is linked to a light chain by a disulfide bond. Monoclonal antibodies may also include chimeric monoclonal antibodies or bispecific antibodies.

Bispecific antibodies (e.g., DuoBody®), heterospecific, heteroconjugate or similar antibodies can also be used that are monoclonal, preferably human or humanized, antibodies that have binding specificities for at least two different antigens. Methods for making bispecific antibodies are known in the art. In one aspect, the recombinant production of bispecific antibodies is based on the co-expression of two immunoglobulin heavy chain-light chain pairs, where the two heavy chains have different specificities (Milstein and Cuello, Nature 1983 Oct. 6-12; 305(5934):537-40). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, can be cumbersome with low product yields and different strategies have been developed to facilitate bispecific antibody production.

As used herein, “antibody” or “antibodies”, include biosimilar antibody molecules approved under the Biologics Price Competition and Innovation Act of 2009 (BPCI Act) and similar laws and regulations globally. Under the BPCI Act, an antibody may be demonstrated to be biosimilar if data show that it is “highly similar” to the reference product notwithstanding minor differences in clinically inactive components and are “expected” to produce the same clinical result as the reference product in terms of safety, purity and potency (Endocrine Practice: February 2018, Vol. 24, No. 2, pp. 195-204). These biosimilar antibody molecules are provided an abbreviated approval pathway, whereby the applicant relies upon the innovator reference product's clinical data to secure regulatory approval. Compared to the original innovator reference antibody that was FDA approved based on successful clinical trials, a biosimilar antibody molecule is referred to herein as a “follow-on biologic”. As presented herein, SIMPONI® (golimumab) is the original innovator reference anti-TNF antibody that was FDA approved based on successful clinical trials. Golimumab has been on sale in the United States since 2009.

As used herein, the terms “sample” or “biologic sample” refer to any biological specimen obtained from a subject or patient. Suitable samples for use in the present invention include, for example, whole blood, plasma, serum, synovial fluid, saliva, urine, stool, tears, any other bodily fluid, tissue samples (e.g., biopsy), and cellular extracts thereof (e.g., red blood cellular extract). In certain embodiments, the sample is a whole blood, plasma or serum. In certain embodiments, the sample is serum. The sample used to determine the level of RF can be the same sample or a different sample than the one used to measure the level of ACPA. For example, a single sample can be used for detecting RF level and ACPA level. In other cases, two samples of the sample type are used for detecting RF and ACPA levels. In certain embodiments, the sample is serum.

As used herein, the terms “treat,” “treating,” or “treatment” refer to an action that reduces the severity or symptoms of the disease or disorder or inhibits the progression or symptoms of the disease or disorder in a patient suffering from the disease or disorder.

As used herein, the terms “subject” and “patient” typically include humans, but can also include other animals such as, e.g., other primates, rodents, canines, felines, and the like. In certain embodiments, the term patient refers to a human patient suffering from and autoimmune disease, e.g., rheumatoid arthritis. In certain embodiments, a patient may be suffering from a viral infection, e.g., COVID-19. It has been reported that IL-6 may play a key role in driving the inflammatory response that leads to morbidity and mortality in patients with COVID-19. It has also been reported that anti-IL-6R antibodies KEVZARA® (sarilumab) and ACTEMRA® (tocilizumab) are entering clinical trials for COVID-19 (Calabrese, Healio Rheumatology, Mar. 19, 2020; Parsons, PMLiVE, Mar. 24, 2020).

### DESCRIPTION

In one aspect, the present invention relates to the finding that the levels of RF or ACPA in a biological sample from a subject are correlated with lower levels of therapeutic IgG exposure. Therefore, in some embodiments, the invention relates to compositions and methods of detecting the level of at least one of RF or ACPA in a biological sample of a subject. In some embodiments, the invention relates to methods of modulating the level or dosage of a therapeutic IgG based upon the presence of an increased level of at least one of RF or ACPA in a biological sample from a subject.

In one embodiment, the therapeutic IgG is a therapeutic agent for the treatment of an inflammatory or autoimmune disease or disorder. In one embodiment, the therapeutic IgG is a therapeutic agent for the treatment of an infectious disease or disorder. In one embodiment, the therapeutic IgG is a therapeutic agent for the treatment of cancer. Exemplary IgG antibodies whose dosages can be modulated according to the methods of the invention include, but are not limited to, golimumab and sirukumab.

In one aspect, the methods generally provide for the detection, measuring, and comparison of a level or pattern of RF or ACPA in a body sample from a subject. In some embodiments, the presence or overexpression of RF or ACPA in the sample obtained from the subject, relative to the level RF or ACPA in a control sample or a reference level, is indicative of a subject at risk of low IgG exposure upon administration of a therapeutic IgG. In some embodiments, the method indicates a subject's responsiveness to a treatment or therapy regimen. In one embodiment, the presence or overexpression of RF or ACPA in the sample obtained from the subject, relative to the level RF or ACPA in a control sample or a reference level, is indicative of a subject who will be unresponsive to a standard therapeutic IgG treatment or therapy regimen.

The present invention further relates, in part, to a method of assessing the prognosis or assessing the effectiveness of a treatment of a disease or disorder in a subject in need thereof. In one aspect, the method comprises assessing the presence or level of RF, ACPA, or a combination thereof, wherein the presence or increased level of RF, ACPA, or the combination thereof is associated with a poor prognosis or a decreased effectiveness of a treatment for the disease or disorder according to the method described herein. In one embodiment, the treatment is an IgG therapeutic.

In some embodiments, one or more additional diagnostic markers may be combined with the RF or ACPA biomarker level to construct models for predicting a patient's response to a standard therapeutic IgG treatment or therapy regimen. For example, clinical factors of relevance may include, but are not limited to, the subject's age, the subject's medical history, the subject's ethnicity, a physical examination, and other biomarkers.

In some embodiments, the invention includes methods of detecting the level of RF, ACPA, or a combination thereof in a biological sample of a subject. The method of the invention may utilize any method known in the art to effectively detect RF. ACPA, or a combination thereof, in a sample. Suitable methods include, but are not limited to, immunoassays, enzyme assays, mass spectrometry, biosensors, and chromatography. Thus, the system of the invention includes the use of any type of instrumentality to detect RF, ACPA, or a combination thereof. In various embodiments of the invention, methods of measuring RF, ACPA, or a combination thereof in a biological sample include, but are not limited to, an immunochromatography assay, an immunodot assay, a Luminex assay, an ELISA assay, an ELISPOT assay, a protein microarray assay, a ligand-receptor binding assay, an immunostaining assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay

In one embodiment, the method may be performed as an affinity-binding assay or immunoassay including the steps of obtaining a sample from the subject, applying the sample to column, bead, surface or support embedded or functionalized with a capture antigen comprising a target antigen of the autoantibody to be detected (e.g., RF or ACPA), contacting the sample with a secondary antibody wherein the secondary antibody is linked to a label or detectable moiety, and detecting the complex formed from the binding of the secondary antibody to the autoantibody of interest. The term “label” may refer to a detectable compound or composition that is conjugated directly or indirectly to a secondary antibody to generate a “labeled” secondary antibody. The label may be detectable by itself (e.g. radioisotope labels or fluorescent labels) or, in the case of an enzymatic label, may catalyze chemical alteration of a substrate compound or composition that is detectable.

In one embodiment, the invention relates to the use of an immunoassay device for detecting RF, ACPA, or a combination thereof in a sample. In various embodiments, the immunoassay device can be used to determine the level of RF, ACPA, or a combination thereof in a sample as compared to a comparator control.

In one embodiment, the method of the invention is an assay for assessing the risk of unresponsiveness to an IgG therapeutic treatment in a subject in need thereof, by determining whether the level of RF or ACPA, or a combination thereof, is increased or decreased in a biological sample obtained from the subject. In various embodiments, to determine whether the level of RF or ACPA, or a combination thereof, is increased or decreased in a biological sample obtained from the subject, the level of RF or ACPA, or a combination thereof, is compared with the level of at least one comparator control, such as a positive control, a negative control, a normal control, a wild-type control, a historical control, a historical norm, or the level of another reference molecule in the biological sample. In some embodiments, the diagnostic assay of the invention is an in vitro assay. In other embodiments, the diagnostic assay of the invention is an in vivo assay.

In certain embodiments, the reference value for RF level can be 10, 11, 12, 13, 14, 15, 16, 17, 18, 19, or 20 IU/mL and the reference value for ACPA level can be 15, 16, 17, 18, 19, 20, 21, 22, 23, 24, or 25 IU/mL.

In certain embodiments, patients positive for RF, ACPA, or RF and ACPA would be administered a therapeutically effective dose of the IgG therapeutic protein that is higher than patients negative for RF, ACPA, or RF and ACPA and/or higher than the recommended dose for standard of care. In certain embodiments the higher therapeutically effective dose of the IgG therapeutic protein is 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% higher than patients negative for RF, ACPA, or RF and ACPA and/or higher than the recommended dose for standard of care. In certain embodiments, patients positive for RF, ACPA, or RF and ACPA would be administered a therapeutically effective dose of the IgG therapeutic protein that is 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% higher than the recommended dose for standard of care.

In certain embodiments, the patients negative for RF, ACPA, or RF and ACPA receive a dose of IgG therapeutic protein that is the standard of care for an approved indication of the IgG therapeutic protein, e.g., an autoimmune disease or infection. In certain embodiments, the patients positive for RF, ACPA, or RF and ACPA would be administered a therapeutically effective dose of the IgG therapeutic protein that is higher than the standard of care for an approved indication of the IgG therapeutic protein, e.g., an autoimmune disease or infection.

In certain embodiments, a therapeutically effective amount of the IgG therapeutic protein may be administered with a suitable pharmaceutical excipient as necessary and can be carried out via any of the accepted modes of administration, e.g., intravenous, topical, subcutaneous, transcutaneous, transdermal, intramuscular, oral, buccal, sublingual, gingival, palatal, intra-joint, parenteral, intra-arteriole, intradermal, intraventricular, intracranial, intraperitoneal, intralesional, intranasal, rectal, vaginal, or by inhalation. A therapeutically effective amount may be administered subcutaneously. Subcutaneous administration of the therapeutically effective amount of the IgG therapeutic protein may be accomplished using a device. The device may be a syringe, a prefilled syringe, an auto-injector, either disposable or reusable, a pen injector, a patch injector, a wearable injector or an ambulatory syringe infusion pump with subcutaneous infusion sets.

In certain embodiments, a therapeutically effective amount of the IgG therapeutic protein may be administered repeatedly, e.g., at least 2, 3, 4, 5, 6, 7, 8, or more times, or the dose may be administered by continuous infusion. The dose may take the form of solid, semi-solid, lyophilized powder, or liquid dosage forms, such as, for example, powders, solutions, suspensions, emulsions, retention enemas, or the like, preferably in unit dosage forms suitable for simple administration of precise dosages.

In certain embodiments, administration of the therapeutically effective amount of IgG therapeutic protein may be repeated after one day, two days, three days, four days, five days, six days, one week, two weeks, three weeks, four weeks, five weeks, six weeks, seven weeks, two months, three months, four months, five months, six months or longer. Repeated courses of treatment are also possible, as is chronic administration. The repeated administration may be at the same dose or at a different dose. For example, the pharmaceutical compositions of the invention may be administered once weekly for eight weeks, followed by once in two weeks for 16 weeks, followed by once in four weeks. In certain embodiments, the therapeutically effective amount of IgG therapeutic protein is administered Q2w (every 2 weeks) or Q4w (every 4 weeks).

### SIMPONI® (Golimumab)

Therapies with anti-TNF agents have been used successfully in the treatment of inflammatory arthritides, but the early anti-TNF agents had limitations with respect to safety, dosing regimen, cost, and/or immunogenicity. To address some of the limitations, a fully human anti-TNF antibody was developed, designated SIMPONI® (golimumab). Golimumab (also known as CNTO 148 and rTNV148B) is a fully human monoclonal antibody with an Immunoglobulin G 1 (IgG1) heavy chain isotype (Glm[z] allotype) and a kappa light chain isotype. The molecular weight of golimumab ranges from 149,802 to 151,064 Daltons.

Golimumab forms high affinity, stable complexes with both the soluble and transmembrane bioactive forms of human tumor necrosis factor alpha (TNFα) with high affinity and specificity which prevents the binding of TNFα to its receptors and neutralizes TNFα bioactivity. No binding to other TNFα superfamily ligands was observed; in particular, golimumab does not bind or neutralize human lymphotoxin. TNFα is synthesized primarily by activated monocytes, macrophages and T cells as a transmembrane protein that self-associates to form a bioactive homotrimer that is rapidly released from the cell surface by proteolysis. The binding of TNFα to either the p55 or p75 TNF receptors leads to clustering of the receptor cytoplasmic domains and initiates signaling. Tumor necrosis factor α has been identified as a key sentinel cytokine that is produced in response to various stimuli and subsequently promotes the inflammatory response through activation of the caspase-dependent apoptosis pathway and the transcription factors nuclear factor (NF)-κB and activator protein-1 (AP-1). Tumor necrosis factor α also modulates the immune response through its role in the organization of immune cells in germinal centers. Elevated expression of TNFα has been linked to chronic inflammatory diseases such as rheumatoid arthritis (RA), as well as spondyloarthropathies such as psoriatic arthritis (PsA) and ankylosing spondylitis (AS). TNFα is an important mediator of the articular inflammation and structural damage that are characteristic of these diseases. For more information about the anti-TNF antibody SIMPONI® (golimumab) and other anti-TNF antibodies, see e.g., U.S. Pat. Nos. 7,250,165; 7,691,378; 7,521,206; 7,815,909; 7,820,169; 8,241,899; 8,603,778; 9,321,836; and 9,828,424.

### Kits

The present invention also pertains to kits useful in the methods of the invention. Such kits comprise various combinations of components useful in any of the methods described elsewhere herein, including for example, materials for identifying at least one antibody, quantitatively analyzing at least one antibody, materials for diagnosing or assessing the risk of unresponsiveness to a therapeutic treatment or therapy regimen based on detection of the antibody, and instructional material. For example, in one embodiment, the kit comprises components useful for the identification of RA or ACPA or a combination thereof in a biological sample. In another embodiment, the kit comprises components useful for the quantification of RA or ACPA or a combination thereof in a biological sample. In a further embodiment, the kit comprises at least one comparator control for determining the presence or level of RA or ACPA or a combination thereof in a biological sample.

In various embodiments, to determine whether the level of an antibody or a target thereof of the invention is present or elevated in a biological sample obtained from the subject, the level of the antibody or the target thereof is compared with the level of at least one comparator contained in the kit, such as a positive control, a negative control, a historical control, a historical norm, or the level of another reference molecule in the biological sample.

### Example Sequences

In certain embodiments, the anti-TNF antibody SIMPONI® (golimumab) comprises the sequences shown below. In certain embodiments, the anti-IL-6 antibody Sirukumab (CNTO 136) comprises the sequences shown below.

### SIMPONI® (Golimumab)

Heavy chain CDRs (HCDRs) and light chain CDRs (LCDRs) are defined by Kabat.

Amino acid sequence of golimumab heavy chain (HC) with CDRs underlined:

(SEQ ID NO: 1)

| | |
|---|---|
| 1 | QVQLVESGGG VVQPGRSLRL SCAASGFIFS SYAMHWVRQA |
| | PGNGLEWVAF MSYDGSNKKY |
| 61 | ADSVKGRFTI SRDNSKNTLY LQMNSLRAED TAVYYCARDR |
| | GIAAGGNYYY YGMDVWGQGT |
| 121 | TVTVSSASTK GPSVFPLAPS SKSTSGGTAA LGCLVKDYFP |
| | EPVTVSWNSG ALTSGVHTFP |
| 181 | AVLQSSGLYS LSSVVTVPSS SLGTQTYICN VNHKPSNTKV |
| | DKKVEPKSCD KTHTCPPCPA |
| 241 | PELLGGPSVF LFPPKPKDTL MISRTPEVTC VVVDVSHEDP |
| | EVKFNWYVDG VEVHNAKTKP |
| 301 | REEQYNSTYR VVSVLTVLHQ DWLNGKEYKC KVSNKALPAP |
| | IEKTISKAKG QPREPQVYTL |
| 361 | PPSRDELTKN QVSLTCLVKG FYPSDIAVEW ESNGQPENNY |
| | KTTPPVLDSD GSFFLYSKLT |
| 421 | VDKSRWQQGN VFSCSVMHEA LHNHYTQKSL SLSPGK |
| 456 | |

Amino acid sequence of golimumab light chain (LC) with CDRs underlined:

(SEQ ID NO: 2)

| | |
|---|---|
| 1 | EIVLTQSPAT LSLSPGERAT LSCRASQSVY SYLAWYQQKP |
| | GQAPRLLIYD ASNRATGIPA |
| 61 | RFSGSGSGTD FTLTISSLEP EDFAVYYCQQ RSNWPPFTFG |
| | PGTKVDIKRT VAAPSVFIFP |
| 121 | PSDEQLKSGT ASVVCLLNNF YPREAKVQWK VDNALQSGNS |
| | QESVTEQDSK DSTYSLSSTL |
| 181 | TLSKADYEKH KVYACEVTHQ GLSSPVTKSF NRGEC |

Amino acid sequence of golimumab variable heavy chain (VH) with CDRs underlined:

(SEQ ID NO: 3)

| | |
|---|---|
| 1 | QVQLVESGGG VVQPGRSLRL SCAASGFIFS SYAMHWVRQA |
| | PGNGLEWVAF MSYDGSNKKY |
| 61 | ADSVKGRFTI SRDNSKNTLY LQMNSLRAED TAVYYCARDR |
| | GIAAGGNYYY YGMDVWGQGT |
| 121 | TVTVSS |

Amino acid sequence of golimumab variable light chain (VL) with CDRs underlined:

(SEQ ID NO: 4)

| | |
|---|---|
| 1 | EIVLTQSPAT LSLSPGERAT LSCRASQSVY SYLAWYQQKP |
| | GQAPRLLIYD ASNRATGIPA |
| 61 | RFSGSGSGTD FTLTISSLEP EDFAVYYCQQ RSNWPPFTFG |
| | PGTKVDIKRT V |

Amino acid sequence of golimumab heavy chain complementarity determining region 1 (HCDR1):

(SEQ ID NO: 5)
SYAMH

Amino acid sequence of golimumab antibody heavy chain complementarity determining region 2 (HCDR2):

(SEQ ID NO: 6)
FMSYDGSNKKYADSVKG

Amino acid sequence of golimumab heavy chain complementarity determining region 3 (HCDR3):

(SEQ ID NO: 7)
DRGIAAGGNYYYYGMDV

Amino acid sequence of golimumab light chain complementarity determining region 1 (LCDR1):

(SEQ ID NO: 8)
RASQSVYSYLA

Amino acid sequence of golimumab light chain complementarity determining region 2 (LCDR2):

(SEQ ID NO: 9)
DASNRAT

Amino acid sequence of golimumab light chain complementarity determining region 3 (LCDRL):

(SEQ ID NO: 10)
QQRSNWPPFT

### Sirukumab (CNTO 136)

Heavy chain CDRs (HCDRs) and light chain CDRs (LCDRs) are defined by Kabat.

Amino acid sequence of sirukumab heavy chain (HC) with CDRs underlined:

(SEQ ID NO: 11)

| | |
|---|---|
| 1 | EVQLVESGGG LVQPGGSLRL SCAASGFTFS PFAMSWVRQA |
| | PGKGLEWVAK ISPGGSWTYY |
| 61 | SDTVTGRFTI SRDNAKNSLY LQMNSLRAED TAVYYCARQL |
| | WGYYALDIWG QGTTVTVSSA |
| 121 | STKGPSVFPL APSSKSTSGG TAALGCLVKD YFPEPVTVSW |
| | NSGALTSGVH TFPAVLQSSG |
| 181 | LYSLSSVVTV PSSSLGTQTY ICNVNHKPSN TKVDKKVEPK |
| | SCDKTHTCPP CPAPELLGGP |
| 241 | SVFLFPPKPK DTLMISRTPE VTCVVVDVSH EDPEVKFNWY |
| | VDGVEVHNAK TKPREEQYNS |
| 301 | TYRVVSVLTV LHQDWLNGKE YKCKVSNKAL PAPIEKTISK |
| | AKGQPREPQV YTLPPSRDEL |
| 361 | TKNQVSLTCL VKGFYPSDIA VEWESNGQPE NNYKTTPPVL |
| | DSDGSFFLYS KLTVDKSRWQ |
| 421 | QGNVFSCSVM HEALHNHYTQ KSLSLSPGK |

Amino acid sequence of sirukumab light chain (LC) with CDRs underlined:

(SEQ ID NO: 12)

| | |
|---|---|
| 1 | EIVLTQSPAT LSLSPGERAT LSCSASISVS YMYWYQQKPG |
| | QAPRLLIYDM SNLASGIPAR |
| 61 | FSGSGSGTDF TLTISSLEPE DFAVYYCMQW SGYPYTFGGG |
| | TKVEIKRTVA APSVFIFPPS |
| 121 | DEQLKSGTAS VVCLLNNFYP REAKVQWKVD NALQSGNSQE |
| | SVTEQDSKDS TYSLSSTLTL |
| 181 | SKADYEKHKV YACEVTHQGL SSPVTKSFNR GEC |

Amino acid sequence of sirukumab variable heavy chain (VH) with CDRs underlined: (SEQ ID NO:13)

| | |
|---|---|
| 1 | EVQLVESGGG LVQPGGSLRL SCAASGFTFS PFAMSWVRQA |
| | PGKGLEWVAK ISPGGSWTYY |
| 61 | SDTVTGRFTI SRDNAKNSLY LQMNSLRAED TAVYYCARQL |
| | WGYYALDIWG QGTTVTVSS |

Amino acid sequence of sirukumab variable light chain (VL) with CDRs underlined: (SEQ ID NO:14)

| | |
|---|---|
| 1 | EIVLTQSPAT LSLSPGERAT LSCSASISVS YMYWYQQKPG |
| | QAPRLLIYDM SNLASGIPAR |
| 61 | FSGSGSGTDF TLTISSLEPE DFAVYYCMQW SGYPYTFGGG |
| | TKVEIKRTV |

Amino acid sequence of sirukumab heavy chain complementarity determining region 1 (HCDR1):

(SEQ ID NO: 15)
PFANS

Amino acid sequence of sirukumab antibody heavy chain complementarity determining region 2 (HCDR2):

(SEQ ID NO: 16)
KISPGGSWTYYSDTVTG

Amino acid sequence of sirukumab heavy chain complementarity determining region 3 (HCDR3):

(SEQ ID NO: 17)
QLWGYYALDI

Amino acid sequence of sirukumab light chain complementarity determining region 1 (LCDR1):

(SEQ ID NO: 18)
SASISVSYMY

Amino acid sequence of sirukumab light chain complementarity determining region 2 (LCDR2): (SEQ ID NO:19)

### DMSNLAS

Amino acid sequence of sirukumab light chain complementarity determining region 3 (LCDRL):

(SEQ ID NO: 20)
MQWSGYPYT

### EXAMPLES

### 1. Pharmacokinetic Effects of Serum RF and ACPA Levels on IgG Therapeutic Proteins

### Data and Methods

Correlation analyses were conducted to explore the relationships between baseline serum RF and ACPA levels and the serum drug concentrations of IgG therapeutic proteins sirukumab and golimumab. Data were pooled from 4 phase 3 trials in patients with active RA, i.e., SIRROUND-D and SIRROUND-T clinical trials with sirukumab (Aletaha et al., Lancet. 2017, 389:1206-1217; Takeuchi et al., Ann Rheum Dis. 2017, 76:2001-2008) and the GO-BEFORE and GO-FORWARD clinical trials with golimumab (Emery et al., Arthritis Care Res (Hoboken). 2013 November; 65(11):1732-42; Genovese et al., J Rheumatol. 2012 June; 39(6):1185-91). Patients in sirukumab Phase 3 studies were enrolled in a dose treatment arm and were allowed concomitant methotrexate therapy while patients in the golimumab Phase 3 studies were enrolled prospectively into treatment arms with or without methotrexate. Distribution of the steady-state serum drug concentrations versus the baseline RF and ACPA status were explored and summarized below for each IgG therapeutic protein.

### Sirukumab Results

Based on the distribution plots and the descriptive analysis of the pooled data for sirukumab, correlations were observed between serum drug concentrations and the serum RF and ACPA levels, wherein patients with higher RF or ACPA levels tended to have lower drug exposures. Lower median drug exposures were also observed for patients who were higher in both RF and ACPA levels compared to the single positive condition.

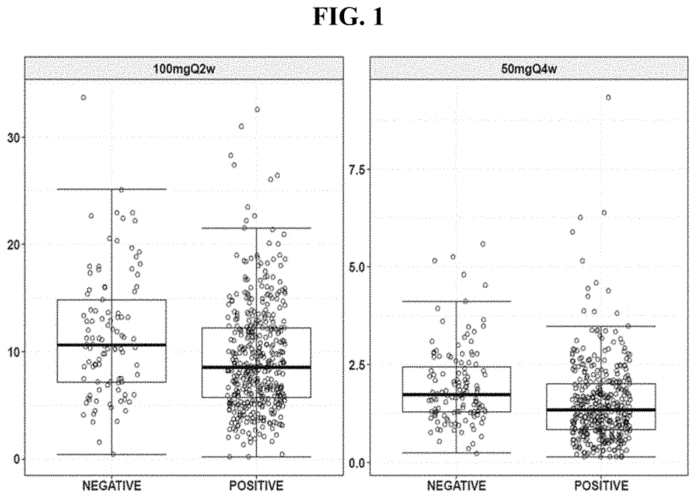

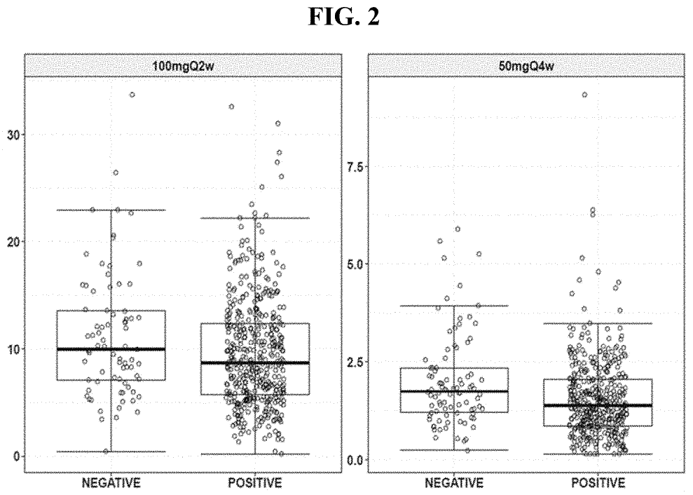

The median serum sirukumab trough concentrations at steady state were approximately 20-22% lower in subjects who were positive for RF (ie., RF ≥15 IU/mL) compared to subjects with negative RF (FIG. 1), and approximately 12-20% lower in subjects who were positive for ACPA (ie, ACPA ≥20 IU/ml) versus subjects with negative ACPA levels (FIG. 2).

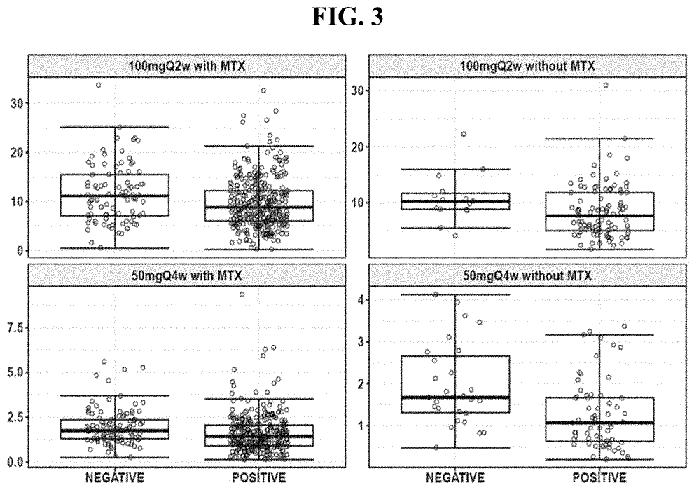

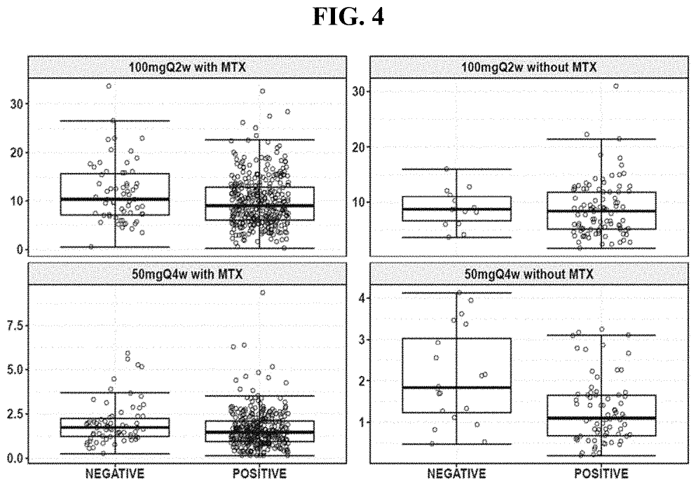

Patients with higher RF or ACPA levels tended to have lower drug exposures regardless of the concomitant methotrexate use (FIG. 3 and FIG. 4). The effect of methotrexate on sirukumab PK was not apparent (Xu Y, et al., J Clin Pharmacol. 2018 July; 58(7):939-951) because most all subjects involved in phase 3 trials were on concomitant methotrexate.

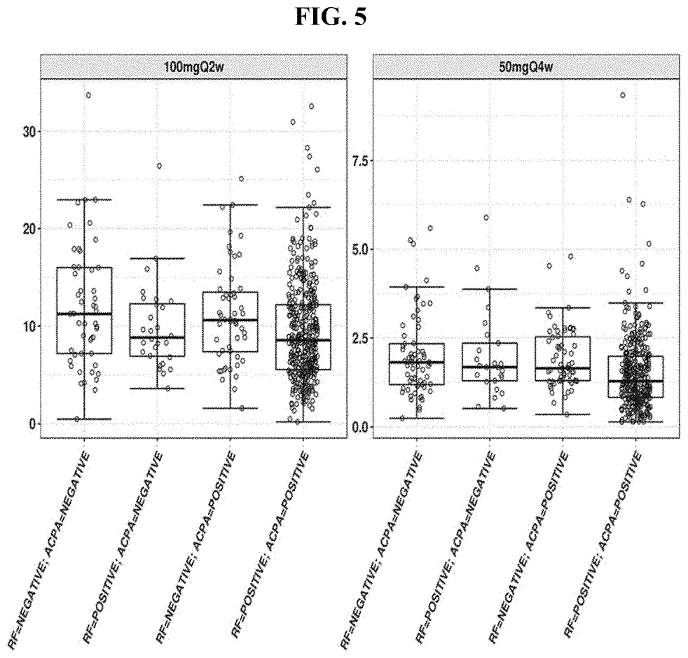

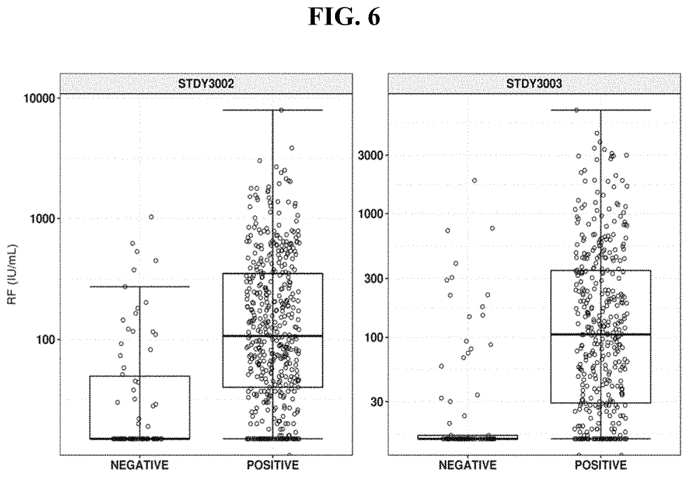

Patients with higher ACPA tended to have higher RF levels in sirukumab trials (FIG. 6). Compared to subjects who were negative for both RF and ACPA, the median serum sirukumab trough concentrations at steady state were approximately 24-29% lower in subjects who were positive for both RF and ACPA, 7-22% lower in RF positive only subjects, and 6-9% lower in ACPA positive subjects. The current data show a trend for a combined RF and ACPA effect on sirukumab PK, but it is noted that results at this time are limited by the small sample size of RF and ACPA double negative or single negative populations, large PK variability, and overlap in PK distribution between groups (FIG. 5).

TABLE 1
Sirukumab Serum Concentrations at Week 16 Grouped
by Baseline Rheumatoid Factor (RF) Status (Positive:
RF ≥ 15 IU/mL vs Negative: RF < 15 IU/mL).

| | | | | | 25^{th} | 75^{th} |
|---|---|---|---|---|---|---|
| Groups | n | Mean | SD | Median | Percentile | Percentile |
| 100 mg Q2w | | | | | | |
| negative | 101 | 11.55 | 5.79 | 10.67 | 7.18 | 14.87 |
| positive | 425 | 9.39 | 5.04 | 8.55 | 5.71 | 12.22 |
| 50 mg Q4w | | | | | | |
| negative | 128 | 1.96 | 1.01 | 1.74 | 1.29 | 2.44 |
| positive | 384 | 1.53 | 1.03 | 1.35 | 0.84 | 2.00 |

Q2w = every 2 weeks; Q4w = every 4 weeks.

TABLE 2
Sirukumab Serum Concentrations at Week 16 Grouped by Baseline
Anti-Citrullinated Protein Antibodies (ACPA) Status (Positive:
ACPA ≥ 20 IU/mL versus Negative: ACPA < 20 IU/mL).

| | | | | | 25^{th} | 75^{th} |
|---|---|---|---|---|---|---|
| Groups | n | Mean | SD | Median | Percentile | Percentile |
| 100 mg Q2w | | | | | | |
| negative | 77 | 11.20 | 5.88 | 9.94 | 7.07 | 13.61 |
| positive | 448 | 9.59 | 5.10 | 8.72 | 5.77 | 12.39 |
| 50 mg Q4w | | | | | | |
| negative | 90 | 2.00 | 1.17 | 1.75 | 1.21 | 2.35 |
| positive | 422 | 1.56 | 1.00 | 1.39 | 0.88 | 2.05 |

Q2w = every 2 weeks; Q4w = every 4 weeks.

TABLE 3
Sirukumab Serum Concentrations at Week 16 Grouped by Baseline
Rheumatoid Factor (RF) Status (Positive: RF ≥ 15
IU/mL vs Negative: RF < 15 IU/mL) and Methotrexate Use.

| | | | | | 25^{th} | 75^{th} |
|---|---|---|---|---|---|---|
| Groups | n | Mean | SD | Median | Percentile | Percentile |
| 100 mg Q2w | | | | | | |
| with MTX | | | | | | |
| negative | 86 | 11.67 | 6.02 | 11.19 | 7.09 | 15.57 |
| positive | 328 | 9.68 | 5.11 | 8.79 | 5.97 | 12.23 |
| 100 mg Q2w | | | | | | |
| without MTX | | | | | | |
| negative | 15 | 10.83 | 4.36 | 10.24 | 8.79 | 11.68 |
| positive | 97 | 8.41 | 4.70 | 7.70 | 5.06 | 11.87 |
| 50 mg Q4w | | | | | | |
| with MTX | | | | | | |
| negative | 101 | 1.95 | 1.01 | 1.75 | 1.29 | 2.35 |
| positive | 319 | 1.59 | 1.07 | 1.42 | 0.88 | 2.04 |
| 50 mg Q4w | | | | | | |
| without MTX | | | | | | |
| negative | 27 | 1.97 | 1.00 | 1.67 | 1.32 | 2.66 |
| positive | 65 | 1.24 | 0.82 | 1.07 | 0.63 | 1.66 |

MTX = Methotrexate; Q2w = every 2 weeks; Q4w = every 4 weeks.

TABLE 4
Sirukumab Serum Concentrations at Week 16 Grouped
by Baseline Anti-Citrullinated Protein Antibodies
(ACPA) Status (Positive: ACPA ≥ 20 IU/mL versus
Negative: ACPA < 20 IU/mL) and Methotrexate Use.

| | | | | | 25^{th} | 75^{th} |
|---|---|---|---|---|---|---|
| Groups | n | Mean | SD | Median | Percentile | Percentile |
| 100 mg Q2w | | | | | | |
| with MTX | | | | | | |
| negative | 63 | 11.71 | 6.20 | 10.31 | 7.12 | 15.60 |
| positive | 350 | 9.83 | 5.14 | 8.99 | 6.00 | 12.90 |
| 100 mg Q2w | | | | | | |
| without MTX | | | | | | |
| negative | 14 | 8.94 | 3.42 | 8.71 | 6.64 | 10.99 |
| positive | 98 | 8.71 | 4.88 | 8.36 | 5.09 | 11.89 |
| 50 mg Q4w | | | | | | |
| with MTX | | | | | | |
| negative | 70 | 1.97 | 1.19 | 1.72 | 1.21 | 2.24 |
| positive | 350 | 1.62 | 1.03 | 1.45 | 0.94 | 2.08 |
| 50 mg Q4w | | | | | | |
| without MTX | | | | | | |
| negative | 20 | 2.09 | 1.15 | 1.83 | 1.23 | 3.03 |
| positive | 72 | 1.28 | 0.78 | 1.10 | 0.67 | 1.66 |

MTX = Methotrexate; Q2w = every 2 weeks; Q4w = every 4 weeks.

TABLE 5
Sirukumab Serum Concentrations at Week 16 Grouped by Baseline
Rheumatoid Factor (RF) Status (Positive: RF ≥ 15
IU/mL vs Negative: RF < 15 IU/mL) and Baseline Anti-
Citrullinated Protein Antibodies (ACPA) Status (Positive:
ACPA ≥ 20 IU/mL versus Negative: ACPA < 20 IU/mL).

| | | | | | 25^{th} | 75^{th} |
|---|---|---|---|---|---|---|
| Groups | n | Mean | SD | Median | Percentile | Percentile |
| 100 mg Q2w | | | | | | |
| with MTX | | | | | | |
| RF negative, | 49 | 11.92 | 6.42 | 11.24 | 7.18 | 16.02 |
| ACPA negative | | | | | | |
| RF positive, | 28 | 9.94 | 4.62 | 8.82 | 6.91 | 12.31 |
| ACPA negative | | | | | | |
| RF negative, | 52 | 11.20 | 5.17 | 10.60 | 7.37 | 13.50 |
| ACPA positive | | | | | | |
| RF positive, | 396 | 9.37 | 5.06 | 8.55 | 5.54 | 12.22 |
| ACPA positive | | | | | | |
| 50 mg Q4w | | | | | | |
| with MTX | | | | | | |
| RF negative, | 65 | 1.99 | 1.15 | 1.81 | 1.19 | 2.34 |
| ACPA negative | | | | | | |
| RF positive, | 25 | 2.02 | 1.26 | 1.68 | 1.30 | 2.35 |
| ACPA negative | | | | | | |
| RF negative, | 63 | 1.92 | 0.84 | 1.65 | 1.30 | 2.53 |
| ACPA positive | | | | | | |
| RF positive, | 359 | 1.50 | 1.01 | 1.28 | 0.83 | 1.98 |
| ACPA positive | | | | | | |

Q2w = every 2 weeks;
Q4w = every 4 weeks

TABLE 6
Baseline Rheumatoid Factor (RF) Levels Grouped by
Baseline Anti-Citrullinated Protein Antibodies (ACPA)
Status (Positive: ACPA ≥ 20 IU/mL versus Negative:
ACPA < 20 IU/mL) in sirukumab Phase 3 Trials.

| | | | | | 25^{th} | 75^{th} |
|---|---|---|---|---|---|---|
| Groups | n | Mean | SD | Median | Percentile | Percentile |
| STDY3002 | | | | | | |
| negative | 74 | 78.09 | 162.15 | 15.00 | 15.00 | 49.50 |
| positive | 470 | 307.54 | 583.28 | 106.50 | 39.25 | 348.50 |
| STDY3003 | | | | | | |
| negative | 93 | 74.53 | 224.86 | 15.00 | 15.00 | 16.00 |
| positive | 400 | 351.74 | 703.62 | 105.00 | 29.00 | 344.00 |
| Total | | | | | | |
| negative | 167 | 76.11 | 198.97 | 15.00 | 15.00 | 31.00 |
| positive | 870 | 327.86 | 641.42 | 105.50 | 35.00 | 346.00 |

STDY3002 = SURROUD-D Trial;
STDY3003 = SURROUD-T Trial

### Golimumab Results

Based on the distribution plots and the descriptive analyses of the pooled data for golimumab, similar trends compared to sirukumab were observed between serum golimumab trough concentrations at steady-state and the baseline RF or ACPA levels.

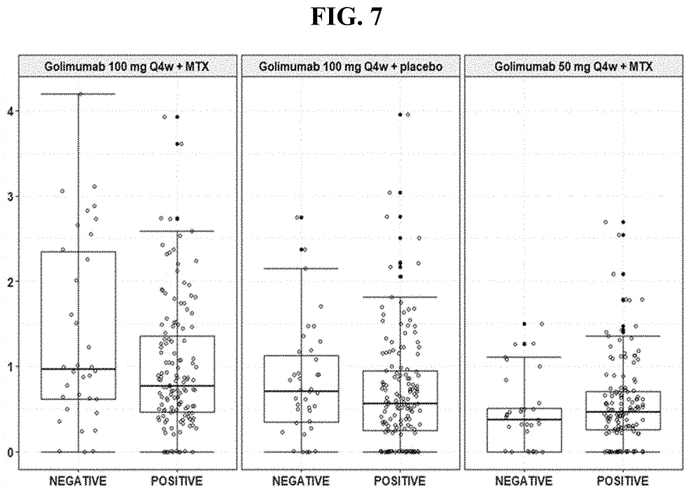

The median serum golimumab trough concentrations at steady-state following treatment with golimumab 100 mg q4w with or without methotrexate were approximately 20-21% lower in subjects who were positive for RF compared to subjects with negative RF, while the concentration following treatment with golimumab 50 mg q4w with methotrexate were 24% higher in RF positive subjects compared to subjects with negative RF (FIG. 7). This unexpected opposite trend is likely due to data variability in the golimumab 50 mg Q4w with methotrexate group.

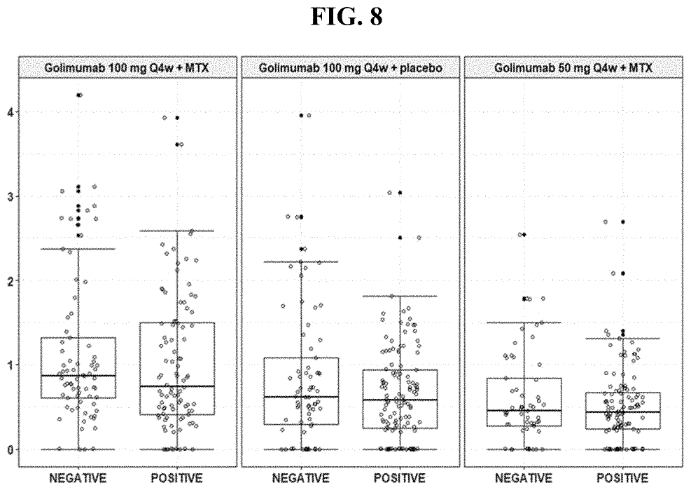

The median golimumab steady-state concentration were approximately 2-14% lower in subjects who were positive for ACPA versus subjects with negative ACPA levels following all 3 treatments (FIG. 8).

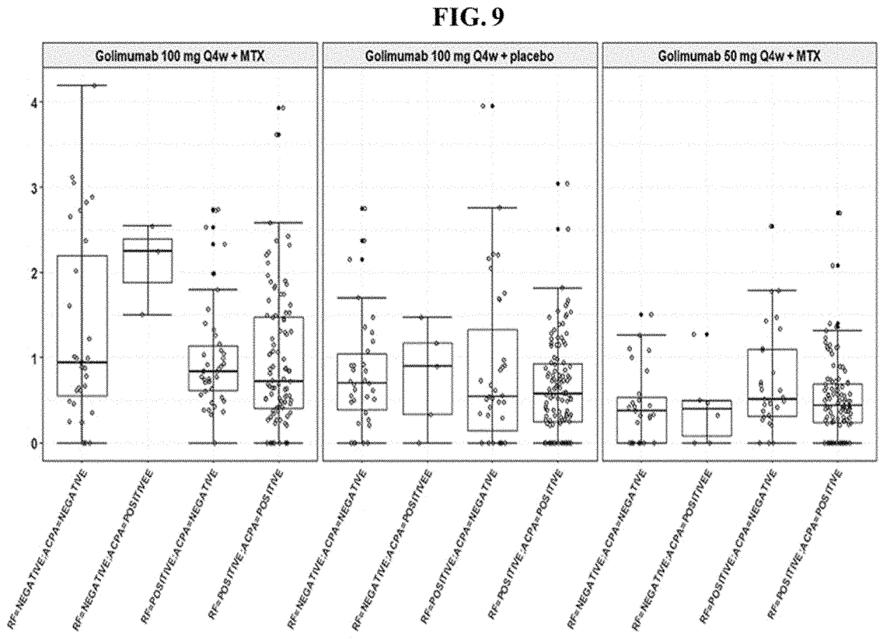

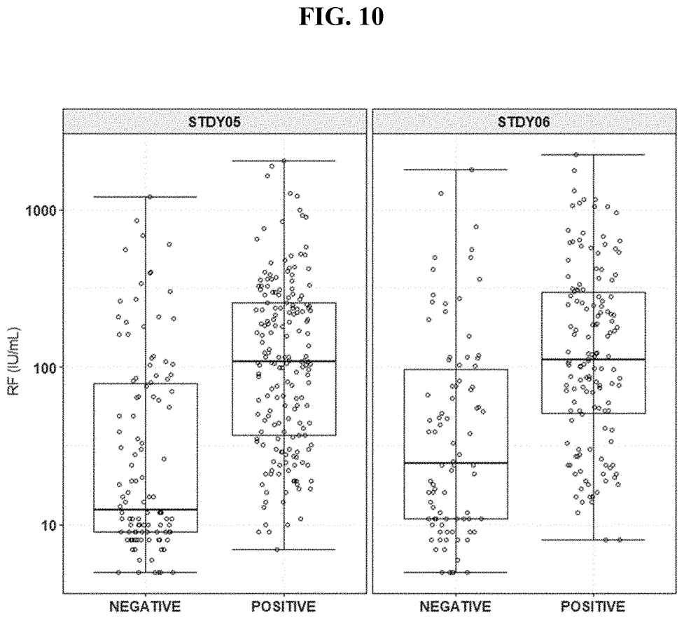

Patients with higher ACPA tended to have higher RF levels in golimumab trials (FIG. 10). For both golimumab 100 mg q4w with or without methotrexate treatment groups compared to subjects who were negative for both RF and ACPA, the median serum golimumab trough concentrations at steady state were approximately 17-23% lower in subjects who were positive for both RF and ACPA and 11-22% lower in RF positive only subjects. The sample sizes of ACPA positive subjects for the golimumab analyses were too small to have meaningful comparison. The median serum golimumab trough concentration following treatment with golimumab 50 mg q4w with methotrexate was 16% higher in subjects who were positive for both RF and ACPA compared to subjects who were negative for both RF and ACPA, which may be attributed to variability (FIG. 9). Note that sample sizes of RF and ACPA double negative or single negative populations were small so conclusions at this time are limited by the small sample size.

TABLE 7
Golimumab Serum Concentrations at Week 24 Grouped by Baseline Rheumatoid
Factor (RF) Status (Positive: RF ≥ 15 IU/mL vs Negative: RF < 15 IU/mL).

| | | | | | 25^{th} | 75^{th} |
|---|---|---|---|---|---|---|
| Groups | n | Mean | SD | Median | Percentile | Percentile |
| 100 mg Q4w + MTX | | | | | | |
| negative | 34 | 1.37 | 1.10 | 0.97 | 0.62 | 2.34 |
| positive | 135 | 0.97 | 0.75 | 0.77 | 0.46 | 1.36 |
| 100 mg Q4w + Placebo | | | | | | |
| negative | 39 | 0.80 | 0.66 | 0.70 | 0.34 | 1.12 |
| positive | 149 | 0.70 | 0.68 | 0.56 | 0.25 | 0.94 |
| 50 mg Q4w + MTX | | | | | | |
| negative | 33 | 0.44 | 0.43 | 0.38 | 0.00 | 0.50 |
| positive | 133 | 0.56 | 0.51 | 0.47 | 0.26 | 0.71 |

Q4w = every 4 weeks.

TABLE 8
Golimumab Serum Concentrations at Week 24 Grouped by Baseline
Anti-Citrullinated Protein Antibodies (ACPA) Status (Positive:
ACPA ≥ 20 IU/mL versus Negative: ACPA < 20 IU/mL).

| | | | | | 25^{th} | 75^{th} |
|---|---|---|---|---|---|---|
| Groups | n | Mean | SD | Median | Percentile | Percentile |
| 100 mg Q4w + MTX | | | | | | |
| negative | 73 | 1.13 | 0.89 | 0.86 | 0.61 | 1.32 |
| positive | 96 | 1.00 | 0.81 | 0.74 | 0.41 | 1.50 |
| 100 mg Q4w + Placebo | | | | | | |
| negative | 69 | 0.84 | 0.82 | 0.62 | 0.29 | 1.08 |
| positive | 119 | 0.65 | 0.56 | 0.58 | 0.25 | 0.94 |
| 50 mg Q4w + MTX | | | | | | |
| negative | 58 | 0.60 | 0.56 | 0.45 | 0.27 | 0.84 |
| positive | 108 | 0.50 | 0.45 | 0.44 | 0.23 | 0.67 |

Q4w = every 4 weeks.

TABLE 9
Golimumab Serum Concentrations at Week 24 Grouped by Baseline Rheumatoid
Factor (RF) Status (Positive: RF ≥ 15 IU/mL vs Negative: RF <
15 IU/mL) and Baseline Anti-Citrullinated Protein Antibodies (ACPA)
Status (Positive: ACPA ≥ 20 IU/mL versus Negative: ACPA < 20 IU/mL).

| | | | | | 25^{th} | 75^{th} |
|---|---|---|---|---|---|---|
| Groups | n | Mean | SD | Median | Percentile | Percentile |
| 100 mg Q4w + MTX | | | | | | |
| RF negative, | 31 | 1.30 | 1.12 | 0.93 | 0.56 | 2.19 |
| ACPA negative | | | | | | |
| RF positive, | 42 | 1.00 | 0.65 | 0.83 | 0.61 | 1.14 |
| ACPA negative | | | | | | |
| RF negative, | 3 | 2.10 | 0.53 | 2.25 | 1.88 | 2.40 |
| ACPA positive | | | | | | |
| RF positive, | 93 | 0.96 | 0.79 | 0.72 | 0.40 | 1.47 |
| ACPA positive | | | | | | |
| 100 mg Q4w + Placebo | | | | | | |
| RF negative, | 34 | 0.81 | 0.68 | 0.69 | 0.39 | 1.04 |
| ACPA negative | | | | | | |
| RF positive, | 35 | 0.87 | 0.95 | 0.54 | 0.15 | 1.32 |
| ACPA negative | | | | | | |
| RF negative, | 5 | 0.78 | 0.60 | 0.90 | 0.33 | 1.17 |
| ACPA positive | | | | | | |
| RF positive, | 114 | 0.65 | 0.56 | 0.57 | 0.25 | 0.93 |
| ACPA positive | | | | | | |
| 50 mg Q4w + MTX | | | | | | |
| RF negative, | 27 | 0.44 | 0.43 | 0.38 | 0.00 | 0.53 |
| ACPA negative | | | | | | |
| RF positive, | 31 | 0.74 | 0.63 | 0.51 | 0.31 | 1.10 |
| ACPA negative | | | | | | |
| RF negative, | 6 | 0.43 | 0.47 | 0.39 | 0.08 | 0.49 |
| ACPA positive | | | | | | |
| RF positive, | 102 | 0.51 | 0.45 | 0.44 | 0.24 | 0.68 |
| ACPA positive | | | | | | |

Q4w = every 4 weeks

TABLE 10
Baseline Rheumatoid Factor (RF) Levels Grouped by
Baseline Anti-Citrullinated Protein Antibodies (ACPA)
Status (Positive: ACPA ≥ 20 IU/mL versus Negative:
ACPA < 20 IU/mL) in golimumab Phase 3 Trials.

| | | | | | 25^{th} | 75^{th} |
|---|---|---|---|---|---|---|
| Groups | n | Mean | SD | Median | Percentile | Percentile |
| STDY05 | | | | | | |
| negative | 114 | 85.39 | 177.90 | 12.50 | 9.00 | 79.00 |
| positive | 181 | 211.34 | 302.75 | 109.00 | 37.00 | 256.00 |
| STDY06 | | | | | | |
| negative | 86 | 119.16 | 264.13 | 24.50 | 11.00 | 97.00 |
| positive | 142 | 253.48 | 350.64 | 111.50 | 50.75 | 297.25 |
| Total | | | | | | |
| negative | 200 | 99.92 | 219.20 | 17.50 | 9.00 | 82.00 |
| positive | 323 | 229.86 | 324.83 | 110.00 | 39.50 | 278.00 |

STDY05 = C0524T05 Trial;
STDY06 = C0524T06 Trial

The analysis revealed trends for the effects of RF and ACPA levels on the PK of IgG therapeutic proteins sirukumab and golimumab, wherein patients with higher RF or higher ACPA levels had lower exposures. In addition, the analysis revealed a trend for a combined RF and ACPA effect on the PK of the IgG therapeutic proteins. The observed trend toward decreased exposure may influence efficacy, thus treatment of patients could possibly be improved with a modified (optimized) therapeutic dose of IgG therapeutic proteins based on the levels of RF, ACPA, or RF and ACPA.

2. Improved Treatments with IgG Therapeutic Proteins Based on Patients' RF, ACPA, or RF and ACPA Levels

Provided herein are methods for improved treatments with IgG therapeutic proteins, e.g., treating a human subject suffering from an autoimmune disease or infection with a modified (optimized) dose of the IgG therapeutic protein based on the levels of RF, ACPA, or RF and ACPA. In addition, disclosed are methods for selecting or recommending a therapeutically effective dose of an IgG therapeutic protein. The methods are based on an association between the levels of rheumatoid factor (RF) and/or anti-cyclic citrullinated protein autoantibodies (ACPA) relative to reference levels and the predicted steady state serum trough concentrations of IgG therapeutic proteins considered for administration for a given approved indication, e.g., an autoimmune disease or infection.

Results presented herein show that an RF level corresponding to being positive for RF (i.e., RF ≥15 IU/mL) indicates a patient with an autoimmune disease (e.g., RA) is likely to have lower exposure to an IgG therapeutic protein (e.g., the human monoclonal antibodies golimumab or sirukumab) than a patient that is negative for RF (i.e., RF<15 IU/mL). Lower exposure in RF positive patients was 20-22% lower median trough concentrations at steady state for sirukumab and 20-21% lower median trough concentrations at steady state for golimumab. Thus, a therapeutically effective amount of the IgG therapeutic protein would be determined to be about 20-22% higher for a patient positive for RF than for a patient negative for RF and/or 20-22% higher than the recommended dose for standard of care.

In certain embodiments, an ACPA level corresponding to being positive for ACPA (i.e., ACPA ≥20 IU/mL) indicates a patient with an autoimmune disease (e.g., RA) is likely to have lower exposure to an IgG therapeutic protein (e.g., the human monoclonal antibodies golimumab or sirukumab) than a patient that is negative for ACPA (i.e., ACPA <20 IU/mL). In certain embodiments, lower exposure in ACPA positive patients is 12-20% lower median trough concentrations at steady state for sirukumab and 2-14% lower median trough concentrations at steady state for golimumab. Thus, a therapeutically effective amount of the IgG therapeutic protein would be determined to be about 2-20% higher for a patient that is positive for ACPA and/or 2-20% higher than the recommended dose for standard of care.

In certain embodiments, RF and ACPA levels corresponding to being positive for both RF (i.e., RF ≥15 IU/mL) and ACPA (i.e., ACPA ≥20 IU/mL) indicates a patient with an autoimmune disease (e.g., RA) is likely to have lower exposure to an IgG therapeutic protein (e.g., the human monoclonal antibodies golimumab or sirukumab) than a patient that is negative for RF (i.e., RF<15 IU/mL) and ACPA (i.e., ACPA <20 IU/mL). In certain embodiments, lower exposure in RF and ACPA positive patients is 24-29% lower median trough concentrations at steady state for sirukumab and 17-23% lower median trough concentrations at steady state for golimumab. Thus, a therapeutically effective amount of the IgG therapeutic protein would be determined to be about 17-29% higher for a patient that is positive for RF and ACPA and/or 17-29% higher than the recommended dose for standard of care.

What is claimed is:

1. A method for predicting exposure of an immunoglobulin G (IgG) therapeutic protein in a human patient in need of treatment with the IgG therapeutic protein, the method comprising:
a.) selecting the human patient in need of treatment with the IgG therapeutic protein;
b.) determining at least one selected from the group consisting of a level of rheumatoid factor (RF) and a level of anti-citrullinated protein antibodies (ACPA) in a biological sample from the patient;
c.) comparing the level of the at least one selected from the group consisting of said RF and said ACPA to a reference value for the at least one selected from the group consisting of said RF and said ACPA;
d.) determining if the patient is positive or negative for at least one selected from the group consisting of said RF and said ACPA, wherein the patient is positive for RF if the level of the RF in the sample is greater than or equal to (≥) the reference value for RF, the patient is positive for ACPA if the level of the ACPA in the sample is ≥the reference value for ACPA, the patient is negative for RF if the level of the RF in the sample is less than (<) the reference value for RF and the patient is negative for ACPA if the level of the ACPA in the sample is <the reference value for ACPA; and
e.) predicting the exposure of the IgG therapeutic protein will be lower in a patient positive for at least one selected from the group consisting of RF and ACPA; and.

2. The method of claim 1, wherein the reference value is 15 international units per milliliter (IU/mL) for RF and the reference value is 20 IU/ml for ACPA.

3. The method of claim 2, wherein the predicted exposure of the IgG therapeutic protein is a predicted steady state serum trough concentration, a predicted steady state serum peak concentration, or a predicted area under the curve concentration-time profile of the IgG therapeutic protein.

4. The method of claim 3, wherein the predicted steady state serum trough concentration, the predicted steady state serum peak concentration, or the predicted area under the curve concentration-time profile of the IgG therapeutic protein is about 20-22% lower in a patient positive for RF compared to a patient negative for RF.

5. The method of claim 3, wherein the predicted steady state serum trough concentration, the predicted steady state serum peak concentration, or the predicted area under the curve concentration-time profile of the IgG therapeutic protein is about 2-20% lower in a patient positive for ACPA compared to a patient negative for ACPA.

6. The method of claim 1, wherein the IgG therapeutic protein is selected from the group consisting of: an anti-tumor necrosis factor (anti-TNF) antibody, and anti-TNF Fc-fusion protein, an anti-interleukin-6 (anti-IL-6) antibody, and an anti-IL-6 receptor (IL-6R) antibody.

7. The method of claim 6, wherein the anti-TNF antibody is selected from the group consisting of SIMPONI® (golimumab), REMICADE® (infliximab), and HUMIRA® (adalimumab).

8. The method of claim 7, wherein the anti-TNF antibody is SIMPONI® (golimumab).

9. The method of claim 6, further comprising the step of treating the patient with the IgG therapeutic protein based on the prediction of exposure.

10. A method for determining a therapeutically effective dose of an IgG therapeutic protein by predicting exposure of the IgG therapeutic protein in a human patient in need of treatment with the IgG therapeutic protein, the method comprising:
a.) selecting the human patient in need of treatment with the IgG therapeutic protein;
b.) determining at least one selected from the group consisting of a level of rheumatoid factor (RF) and a level of anti-citrullinated protein antibodies (ACPA) in a biological sample from the patient;
c.) comparing the level of the at least one selected from the group consisting of said RF and said ACPA to a reference value for the at least one selected from the group consisting of said RF and said ACPA;
d.) determining if the patient is positive or negative for at least one selected from the group consisting of said RF and said ACPA, wherein the patient is positive for RF if the level of the RF in the sample is greater than or equal to (≥) the reference value for RF, the patient is positive for ACPA if the level of the ACPA in the sample is ≥the reference value for ACPA, the patient is negative for RF if the level of the RF in the sample is less than (<) the reference value for RF and the patient is negative for ACPA if the level of the ACPA in the sample is <the reference value for ACPA;
e.) predicting the exposure of the IgG therapeutic protein will be lower in a patient positive for at least one selected from the group consisting of RF and ACPA;
f.) determining the therapeutically effective dose of the IgG therapeutic protein should be administered at a higher dose in a patient positive for at least one selected from the group consisting of RF and ACPA compared to a patient negative for at least one selected from the group consisting of RF and ACPA; and
g.) treating the patient with the therapeutically effective dose of the IgG therapeutic protein.

11. The method of claim 10, wherein the reference value is 15 international units per milliliter (IU/mL) for RF and the reference value is 20 IU/ml for ACPA.

12. The method of claim 11, wherein the predicted exposure of the IgG therapeutic protein is a predicted steady state serum trough concentration, a predicted steady state serum trough concentration, or a predicted area under the curve concentration-time profile of the IgG therapeutic protein.

13. The method of claim 12, wherein it is determined that the therapeutically effective dose of the IgG therapeutic protein should be administered at a dose about 20-22% higher in a patient positive for RF compared to a patient negative for RF, and wherein the patient negative for RF receives the recommended therapeutic dose for the IgG therapeutic protein.

14. The method of claim 12, wherein it is determined that the therapeutically effective dose of the IgG therapeutic protein should be administered at a dose about 2-20% higher in a patient positive for ACPA compared to a patient negative for ACPA, and wherein the patient negative for ACPA receives the recommended therapeutic dose for the IgG therapeutic protein.

15. The method of claim 10, wherein the IgG therapeutic protein is selected from the group consisting of: an anti-tumor necrosis factor (anti-TNF) antibody, and anti-TNF Fc-fusion protein, an anti-interleukin-6 (anti-IL-6) antibody, and an anti-IL-6 receptor (IL-6R) antibody.

16. The method of claim 15, wherein the anti-TNF antibody is selected from the group consisting of SIMPONI® (golimumab), REMICADE® (infliximab), and HUMIRA® (adalimumab).

17. The method of claim 16, wherein the anti-TNF antibody is SIMPONI® (golimumab).
